(19) United States
(12) Patent Application Publication
(10) Pub. No.: US 2003/0212104 A1
(43) Pub. Date: Nov. 13, 2003

(54) Treatment of diabetes and diabetic complications with NHE-1 inhibitors

(75) Inventors: W. Ross Tracey, Niantic, CT (US); Judith L. Treadway, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/428,521

(22) Filed: May 1, 2003

Related U.S. Application Data
(60) Provisional application No. 60/380,028, filed on May 2, 2002.

(51) Int. Cl. A61K 38/28; A61K 38/17; A61K 38/22; A61K 31/573; A61K 31/4709; A61K 31/4152; A61K 31/175; A61K 31/155

(52) U.S. Cl. 514/314; 514/406; 514/3; 514/12; 514/171; 514/635; 514/342; 514/369; 514/592

(57) ABSTRACT

This invention relates to methods of treating or preventing type 2 diabetes, diabetic neuropathy, diabetic cardiomyopathy, cataracts, diabetic retinopathy, foot ulcers, diabetic microangiopathy, diabetic macroangiopathy, diabetic ischemia reperfusion injury, diabetic cardiac ischemia reperfusion injury and/or insulin resistance syndrome (IRS) in mammals, particularly in humans, by administering a sodium-hydrogen exchanger type 1 (NHE-1) inhibitor or a pharmaceutical composition containing such an inhibitor. This invention also relates to combinations comprising NHE-1 inhibitors and a second pharmaceutical agent, said combinations being useful in treating type 2 diabetes, IRS, diabetic neuropathy, diabetic cardiomyopathy, cataracts, diabetic retinopathy, foot ulcers, diabetic ischemia reperfusion injury, diabetic cardiac ischemia reperfusion injury, diabetic microangiopathy and/or diabetic macroangiopathy.

### CROSS REFERENCE TO RELATED APPLICATION

This application claims the benefit of U.S. Provisional Application No. 60/380,028 filed May 2, 2002.

### BACKGROUND OF INVENTION

This invention relates to methods of treating or preventing type 2 diabetes, diabetic neuropathy, diabetic cardiomyopathy, cataracts, diabetic retinopathy, foot ulcers, diabetic microangiopathy, diabetic macroangiopathy, diabetic ischemia reperfusion injury, diabetic cardiac ischemia reperfusion injury and/or insulin resistance syndrome (IRS) in mammals, particularly in humans, by administering a sodium-hydrogen exchanger type 1 (NHE-1) inhibitor or a pharmaceutical composition containing such an inhibitor. This invention also relates to combinations comprising NHE-1 inhibitors and a second pharmaceutical agent, said combinations being useful in treating type 2 diabetes, IRS, diabetic neuropathy, diabetic cardiomyopathy, cataracts, diabetic retinopathy, foot ulcers, diabetic ischemia reperfusion injury, diabetic cardiac ischemia reperfusion injury, diabetic microangiopathy and/or diabetic macroangiopathy.

The diabetic disease state is characterized by an impaired glucose metabolism that manifests itself in, inter alia, elevated blood glucose levels in patients suffering therefrom. Generally, diabetes is classified into two distinct subgroups:

(1) Type 1 diabetes, or insulin-dependent diabetes mellitus (IDDM), which arises when patients lack insulin-producing β-cells in their pancreatic glands, and (2) Type 2 diabetes, or non-insulin dependent diabetes mellitus (NIDDM), which occurs in patients with, inter alia, impaired β-cell function.

At present, Type 1 diabetic patients are treated with insulin, while the majority of Type 2 diabetic patients are treated with hypoglycemic agents, such as sulfonylureas that stimulate β-cell function, with other agents that enhance the tissue sensitivity of the patients towards insulin, or with insulin itself. Although hypoglycemic agents such as sulfonylureas have been employed widely in the treatment of NIDDM, this treatment is, in many instances, not completely satisfactory. In a large number of NIDDM patients, sulfonylureas have proven ineffective in normalizing blood sugar levels of patients, thereby leading to an increased risk of acquiring diabetic complications. Also, many patients gradually lose the ability to respond to treatment with sulfonylureas and are thus gradually forced into insulin treatment. This shift of patients from oral hypoglycemic agent therapy to insulin therapy is usually ascribed to exhaustion of the β-cells in NIDDM patients.

Type 2 diabetes is a heterogeneous disorder which appears to be polygenic in nature. The primary defect that leads to the clinically diagnosed state of Type 2 diabetes is not clearly identified at this time. It is suspected to be due to a defect in one or more of three primary loci—the liver, the beta cell (pancreatic islets), and/or peripheral insulin-responsive tissues (muscle and fat). There is great scientific debate about the primary importance in one loci over others in the etiology and progression of the disease from the non-diseased state, and at least one reference suggests that the primary defect could occur in muscle or adipose tissue, causing reduced insulin stimulated glucose clearance and metabolism, which in turn stiumlates hyperinsulinemia by the beta-cell, increased peripheral insulin resistance and dysregulation of hepatic glucose production.

This progression could then account for a primary defect in peripheral glucose utilization to produce secondary pancreatic and hepatic dysfunction, or coupled with existing subthreshold defects at any of the three loci, to lead from a non-diseased state, to a state of insulin resistance, and/or impaired glucose tolerance without full presentation of Type 2 diabetes. This is called an insulin-resistant state, a Syndrome X state, or a metabolic syndrome state, or a prediabetic state, including, but not limited to, polycystic ovary syndrome, pregnancy, growth hormone disorders, androgen disorders, and the like. Any of the above conditions could progress to worsen glycemic control to the extent that clinical presentation of Type 2 diabetes could result.

For this reason, it is hypothesized that treatment of individuals “at risk” for the onset or progression of phenotype to the state of Type 2 diabetes, before reaching the state at which Type 2 diabetes clinically presents, with a NHE-1 inhibitor to maintain glycemic control and insulin sensitivity, and hence prevent or slow the onset or progression to a clinically diagnosed Type 2 diabetic state, would be useful. Therefore, NHE-1 inhibitors are useful for maintaining glycemic control and/or reducing insulin resistance in patients in whom impaired glucose tolerance or Type 2 diabetes has not presented and who are at increased risk of developing this disease, and/or preventing the disease in patients (people) “at risk” for Type 2 diabetes.

Since many existing forms of diabetes therapy have proven ineffective in achieving completely satisfactory glycemic control, there continues to be a great demand for novel therapeutic approaches.

IRS, as defined herein, means the concomitant existence in a subject of two or more of: hyperinsulinemia, dyslipidemia, hypertension, type 2 diabetes or impaired glucose tolerance, hyperuricemia or gout, a pro-coagulant state, atherosclerosis and/or truncal obesity. At the center of IRS, also known as “Syndrome X” and “Metabolic Syndrome” in the biomedical literature, is the common feature of tissue resistance to the action of insulin. This impaired biological response to insulin is manifested in the metabolic and vascular effects of insulin. Although there are monogenic syndromes of insulin resistance (IR), in which a definite gene has been identified as the cause of insulin resistance (such as leprechaunism), these are relatively rare. By contrast, the more common presentation of the IRS is associated with obesity (particularly abdominal) and appears to be polygenic.

The adaptive response to IR in individuals having IRS produces compensatory hyperinsulinaemia. As subjects with IRS become progressively insulin resistant, they manifest varying degrees of change in clinical parameters, including blood pressure, and/or increased levels of serum glucose, and/or cholesterol and/or triglycerides, and/or uric acid, and/or free fatty acids and/or factors that increase coagulation.

Once these clinical parameters have changed enough, the patient with IRS may differentially manifest well-recognized clinical conditions or diagnoses. These conditions include: type 2 diabetes, hypertension (high blood pressure), hyperlipidemia or dyslipidemia, particularly (but not limited to) hypertriglyceridemia, hyperuricemia or gout, and hypercoagulability (defined as an abnormal, increased tendency for clots to form, particularly inside blood vessels). These clinical conditions are well-recognized risk factors for cardiovascular (coronary artery and cerebrovascular) disease.

While it is difficult to estimate the prevalence of IRS in the general populace due to both the diversity of the collective risk factors associated with the syndrome and the likelihood that many individuals affected by IRS go undetected because they may exhibit no exterior symptoms and have no prior diagnosis of coronary heart disease, it is postulated that at minimum the patient population at risk for the development of IRS includes individuals with obesity, particularly truncal (abdominal) obesity. Obesity is an extremely common problem in the industrialized world and is associated with the clinical conditions mentioned above. Thus, it is very likely that the prevalence of IRS is very high. Considering this potential patient group alone forms an immense population potentially at risk for the development of complications of IRS. For example in the United States in 1994, 23% of the population aged between 20 and 74 had hypertension, which accounted for 5 deaths per 100,000 population (1997). There will be an estimated 154,392,000 patients with diabetes world-wide in the year 2000. Of these, 15,000,000 will be in the US and 934,000 in the UK. The burden of disease for ischaemic heart disease for both sexes in the WHO region estimated for 1998 was 51,948,000 with a mortality of 7,375,000, constituting 13.7% of total mortality and ranking the highest in the mortality score. The burden of diabetes in both sexes in the WHO region estimated for 1998 was 11,668,000. Thus there exists a large medical need for an effective and safe oral therapy for the treatment of IRS and prevention of the development of IRS and its clinical consequences.

Inhibition of NHE-1 in patients with IRS will result in improved insulin sensitivity and action. Enhancing insulin sensitivity improves clinical parameters of IRS results, inter alia, in:

1. Blood glucose control: In patients with type 2 diabetes or impaired glucose tolerance, an improvement in insulin sensitivity results in a decrease in plasma glucose concentrations (either fasting or after an oral glucose tolerance test or a meal). In a related manner, as regulated by the patient's pathophysiology, there will be an improvement in serum insulin concentrations in either the fasting state or after a glucose load or meal. These improvements in blood glucose control, should the subjects have type 2 diabetes, manifest as improvements in measures of long-term blood glucose control, such as, but not limited to, hemoglobin Alc (glycosylated hemoglobin) or fructosamine.

2. Blood pressure: It is believed that improvement in insulin sensitivity yields improvements in both systolic and diastolic blood pressure.

3. Lipids: Improvement in insulin resistance yields improvements in serum lipids, including, but not limited to, serum cholesterol, triglycerides and free fatty acids.

4. Uric Acid: Improvement in insulin resistance yields improvements in serum uric acid.

5. Coagulation Factors: It is believed that improvement in insulin resistance restores normal factors that worsen the procoagulant state.

International Patent Application No. PCT/JP97/04650, published on Jun. 25, 1998 discloses N-[(substituted five-membered heteroaryl)carbonyl]guanidine compounds which are stated to be useful as inhibitors of Na⁺/H⁺ exchange and consequently effective for the treatment of various diseases such as hypertension, arrhythmia, angina pectoris, myocardial infarct, arteriosclerosis, and complications of diabetes.

International Patent Application No. PCT/IB99/00206, published Sep. 02, 1999, discloses compounds of Formula I

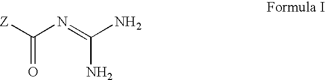

prodrugs thereof and pharmaceutically acceptable salts of said compounds and said prodrugs wherein the definition Z is set forth therein.

International Patent Application No. PCT/EP99/08795, published June 02, 2000, discloses benzoylguanidines of the formula

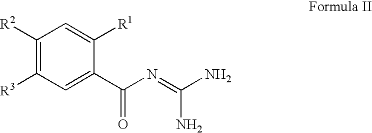

are useful in the treatment of non-insulin-dependent diabetes mellitus.

### SUMMARY OF THE INVENTION

This invention is directed to methods of treating type 2 diabetes, IRS, diabetic neuropathy, diabetic cardiomyopathy, diabetic retinopathy, diabetic microangiopathy, diabetic macroangiopathy, foot ulcers, diabetic ischemia reperfusion injury, diabetic cardiac ischemia reperfusion injury and/or cataracts in a mammal suffering from type 2 diabetes, IRS, diabetic neuropathy, diabetic cardiomyopathy, diabetic retinopathy, diabetic microangiopathy, diabetic macroangiopathy, foot ulcers, diabetic ischemia reperfusion injury, diabetic cardiac ischemia reperfusion injury and/or cataracts comprising administering to said mammal an effective amount of a compound of Formula I

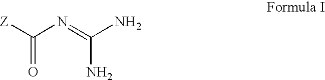

a prodrug or solvate thereof or a pharmaceutically acceptable salt of said compound, prodrug or solvate, wherein

Z is carbon connected and is a five-membered, diaza, diunsaturated ring having two contiguous nitrogens, said ring optionally mono-, di-, or tri-substituted with up to three substituents independently selected from R¹, R² and R³;

or

Z is carbon connected and is a five-membered, triaza, diunsaturated ring, said ring optionally mono- or di-substituted with up to two substituents independently selected from R⁴ and R⁵;

wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N-or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;

wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,

wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines.

Preferably, this invention is directed to methods of treating type 2 diabetes in a mammal comprising administering to said mammal an effective amount of a compound of Formula I as set forth above, a solvate thereof or a pharmaceutically acceptable salt of said compound, said prodrug or said solvate.

Preferably, this invention is also directed to methods of treating IRS in a mammal comprising administering to said mammal an effective amount of a compound of Formula I as set forth above, a solvate thereof or a pharmaceutically acceptable salt of said compound, said prodrug or said solvate.

This invention is also preferably directed to methods of treating one or more of diabetic neuropathy, diabetic cardiomyopathy, diabetic retinopathy, cataracts, foot ulcers, diabetic ischemia reperfusion injury, diabetic cardiac ischemia reperfusion injury, diabetic macroangiopathy or diabetic microangiopathy in a mammal comprising administering to said mammal an effective amount of a compound of Formula I as set forth above, a prodrug or solvate thereof or a pharmaceutically acceptable salt of said compound, prodrug or solvate.

The present invention also provides methods of treating prophylactically an individual in whom diabetic neuropathy, diabetic cardiomyopathy, diabetic retinopathy, cataracts, foot ulcers, diabetic ischemia reperfusion injury, diabetic cardiac ischemia reperfusion injury, diabetic macroangiopathy or diabetic microangiopathy has not yet presented, but in whom there is an increased risk of developing one or more of such conditions, which methods comprise administering to an individual in need thereof an effective amount of a compound of Formula I as set forth above, a prodrug or solvate thereof or a pharmaceutically acceptable salt of said compound, prodrug or solvate.

The present invention also provides methods of treating prophylactically an individual in whom Type 2 diabetes mellitus has not yet presented, but in whom there is an increased risk of developing such condition, which methods comprise administering to an individual in need thereof an effective amount of a compound of Formula I as set forth above, a prodrug or solvate thereof or a pharmaceutically acceptable salt of said compound, prodrug or solvate.

In the methods and kits of this invention, the following compounds, prodrugs and solvates thereof and pharmaceutically acceptable salts of said compounds, prodrugs and solvates are particularly preferred:

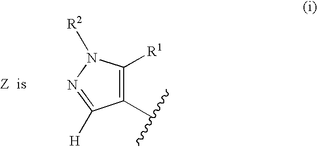

wherein R¹ is (C₃-C₇)cycloalkyl, phenyl or phenyl(C₁-C₄)alkyl, said (C₃-C₇)cycloalkyl optionally substituted with from one to three fluorines, said R¹ substituent optionally mono- or di-substituted independently with (C₁-C₄)alkoxy, (C₁-C₄)alkylthio, (C₁-C₄)alkylsulfinyl or (C₁-C₄)alkylsulfonyl; and R² is (C₁-C₄)alkyl, (C₃-C₄)cycloalkyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;

wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,

wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; or

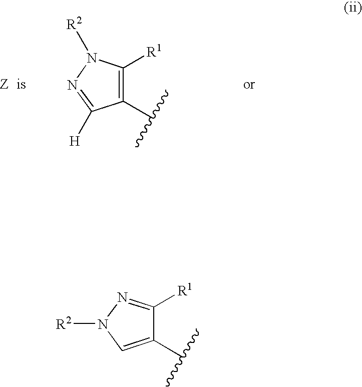

wherein R¹ is (C₁-C₄)alkyl, (C₃-C₇)cycloalkyl, phenyl or phenyl(C₁-C₄)alkyl, said (C₁-C₄)alkyl optionally substituted with from one to nine fluorines, said R¹ substituent optionally mono- or di-substituted independently with (C, -C₄)alkoxy, (C, -C₄)alkylthio, (C₁-C₄)alkylsulfinyl or (C, -C₄)alkylsulfonyl; and

R² is a five to six membered nonaromatic heterocyclic ring having one to two heteroatoms selected independently from nitrogen, sulfur and oxygen or R² is unsubstituted (C₁-C₄)alkyl or unsubstituted (C₃-C₇)cycloalkyl; or R² is phenyl(C₁-C₄)alkyl, or a bicyclic ring consisting of two fused five and/or six membered partially saturated, fully saturated or fully unsaturated rings taken independently having one to four heteroatoms selected independently from nitrogen, sulfur and oxygen, said R² substituents optionally substituted on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,

wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C, -C₄)alkanoylamino, (C, -C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines, or

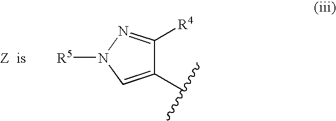

wherein R⁴ is (C₁-C₄)alkyl, (C₃-C₇)cycloalkyl, phenyl or phenyl(C₁-C₄)alkyl, said (C₁-C₄)alkyl optionally substituted with from one to nine fluorines, said R⁴ substituent optionally mono- or di-substituted independently with (C, -C₄)alkoxy, (C₁-C₄)alkylthio, (C, -C₄)alkylsulfinyl or (C, -C₄)alkylsulfonyl; and

R⁵ is a five to six membered nonaromatic heterocyclic ring having one to two heteroatoms selected independently from nitrogen, sulfur and oxygen or R⁵ is unsubstituted (C₁-C₄)alkyl or unsubstituted (C₃-C₇)cycloalkyl; or R⁵ is phenyl(C₁-C₄)alkyl, or a bicyclic ring consisting of two fused five and/or six membered partially saturated, fully saturated or fully unsaturated rings taken independently having one to four heteroatoms selected independently from nitrogen, sulfur and oxygen, said R⁵ substituents optionally substituted on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,

wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R ⁸substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; or

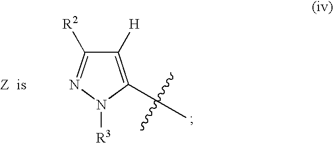

wherein R² is (C₁-C₄)alkyl, (C₃-C₇)cycloalkyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;

wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)aikanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,

wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; and

R³ is (C₁-C₄)alkyl, (C₃-C₇)cycloalkyl, phenyl or phenyl(C₁-C₄)alkyl, said (C₁-C₄)alkyl optionally substituted with from one to nine fluorines, said R substituent optionally mono- or di-substituted independently with (C₁-C₄)alkoxy, (C₁-C₄)alkylthio, (C₁-C₄)alkylsulfinyl, (C₁-C₄)alkylsulfonyl or

(C₁-C₄)alkyl with the proviso that no individual ring of a bicyclic ring can have more than three heteroatoms within the ring.

In the methods and kits of this invention, the following compounds, prodrugs thereof, solvates thereof and pharmaceutically acceptable salts of said compounds, prodrugs and solvates, are also particularly preferred:

[5-methyl-1-(quinolin-6-yl)-1H-pyrazole-4-carbonyl]guanidine;

[5-methyl-i-(naphthalen-1-yl)-1H-pyrazole-4-carbonyl]guanidine;

[5-cyclopropyl-1-(quinolin-5-yl)-1H-pyrazole-4-carbonyl]guanidine;

[5-cyclopropyl-1-(quinolin-8-yl)-1H-pyrazole-4-carbonyl]guanidine;

[5-methyl-2-phenyl-2H-1,2,3-triazole-4-carbonyl]guanidine;

[5-methyl-2-(3-methoxyphenyl)-2H-1 ,2,3-triazole-4-carbonyl]guanidine;

[2-(3-bromophenyl)-5-methyl-2H-1,2,3-triazole-4-carbonyl]guanidine;

[5-cyclopropyl-1-(2-trifluoromethylphenyl)-1H-pyrazole-4-carbonyl]guanidine;

[5-cyclopropyl-1-phenyl-1H-pyrazole-4-carbonyl]guanidine;

[5-cyclopropyl-1-(2,6-dichlorophenyl)-1H-pyrazole-4-carbonyl]guanidine;

[1-(2-chloro-4-methylsulfonylphenyl)-5-cyclopropyl-1H-pyrazole-4-carbonyl]guanidine;

[1-(2-chlorophenyl)-5-cyclopropyl-1H-pyrazole-4-carbonyl]guanidine;

[1-(2-trifluoromethyl-4-fluorophenyl)-5-cyclopropyl-1H-pyrazole-4-carbonyl]guanidine;

[1-(2-bromophenyl)-5-cyclopropyl-1H-pyrazole-4-carbonyl]guanidine;

[1-(2-fluorophenyl)-5-cyclopropyl-1H-pyrazole-4-carbonyl]guanidine;

[1-(2-chloro-5-methoxyphenyl)-5-cyclopropyl-1H-pyrazole-4-carbonyl]guanidine;

[1-(2-chloro-4-methylaminosulfonylphenyl)-5-cyclopropyl-1H-pyrazole-4-carbonyl]guanidine;

[1-(2,5-dichlorophenyl)-5-cyclopropyl-1H-pyrazole-4-carbonyl]guanidine;

[1-(2,3-dichlorophenyl)-5-cyclopropyl-1H-pyrazole-4-carbonyl]guanidine;

[1-(2-chloro-5-aminocarbonylphenyl)-5-cyclopropyl-1H-pyrazole-4-carbonyl]guanidine;

[1-(2-chloro-5-aminosulfonylphenyl)-5-cyclopropyl-1H-pyrazole-4-carbonyl]guanidine;

[1-(2-fluoro-6-trifluoromethylphenyl)-5-cyclopropyl-1H-pyrazole-4-carbonyl]guanidine;

[1-(2-chloro-5-methylsulfonylphenyl)-5-cyclopropyl-1H-pyrazole-4-carbonyl]guanidine;

[1-(2-chloro-5-dimethylaminosulfonylphenyl)-5-cyclopropyl-1H-pyrazole-4-carbonyl]guanidine;

[1-(2-trifluoromethyl-4-chlorophenyl)-5-cyclopropyl-1H-pyrazole-4-carbonyl]guanidine;

[1-(8-bromoquinolin-5-yl)-5-cyclopropyl-1H-pyrazole-4-carbonyl]guanidine;

[1-(6-chloroquinolin-5-yl)-5-cyclopropyl-1H-pyrazole-4-carbonyl]guanidine;

[1-(indazol-7-yI)-5-cyclopropyl-1H-pyrazole-4-carbonyl]guanidine;

[1-(benzimidazol-5-yl)-5-cyclopropyl-1H-pyrazole-4-carbonyl]guanidine;

[1-(1-isoquinolyl)-5-cyclopropyl-1H-pyrazole-4-carbonyl]guanidine;

[5-cyclopropyl-1-(4-quinolinyl)-1H-pyrazole-4-carbonyl]guanidine;

[1-(indazol-6-yl)-5-ethyl-1H-pyrazole-4-carbonyl]guanidine;

[1-(indazol-5-yI)-5-ethyl-1H-pyrazole-4-carbonyl]guanidine;

[1-(benzimidazol-5-yl)-5-ethyl-1H-pyrazole-4-carbonyl]guanidine;

[1-(1-methylbenzimidazol-6-yl)-5-ethyl-1H-pyrazole-4-carbonyl]guanidine

[1-(5-quinolinyl)-5-n-propyl-1H-pyrazole-4-carbonyl]guanidine;

[1-(5-quinolinyl)-5-isopropyl-1H-pyrazole-4-carbonyl]guanidine;

[5-ethyl-1-(6-quinolinyl)-1H-pyrazole-4-carbonyl]guanidine;

[1-(2-methylbenzimidazol-5-yl)-5-ethyl-1H-pyrazole-4-carbonyl]guanidine;

[1-(1 ,4-benzodioxan-6-yl)-5-ethyl-1H-pyrazole-4-carbonyl]guanidine;

[1-(benzotriazol-5-yl)-5-ethyl-1H-pyrazole-4-carbonyl]guanidine;

[1-(3-chloroindazol-5-yl)-5-ethyl-1H-pyrazole-4-carbonyl]guanidine;

[1-(5-quinolinyl)-5-butyl-1H-pyrazole-4-carbonyl]guanidine;

[5-propyl-1-(6-quinolinyl)-1H-pyrazole-4-carbonyl]guanidine;

[5-isopropyl-1-(6-quinolinyl)-1H-pyrazole-4-carbonyl]guanidine;

[1-(2-chlorophenyl)-5-methyl-1H-pyrazole-4-carbonyl]guanidine;

[5-methyl-1-(2-trifluoromethylphenyl)-1H-pyrazole-4-carbonyl]guanidine;

[5-ethyl-1-phenyl-1H-pyrazole-4-carbonyl]guanidine;

[5-cyclopropyl-1-(2-trifluoromethylphenyl)-1H-pyrazole-4-carbonyl]guanidine;

[5-cyclopropyl-1-phenyl-1H-pyrazole-4-carbonyl]guanidine; or

[5-cyclopropyl-1-(2,6-dichlorophenyl)-1H-pyrazole-4-carbonyl]guanidine.

In the methods and kits of this invention, the following compounds, prodrugs thereof, solvates thereof and pharmaceutically acceptable salts of said compounds, prodrugs and solvates, are still more particularly preferred:

[5-cyclopropyl-1-(quinolin-5-yl)-1H-pyrazole-4-carbonyl]guanidine; [5-cyclopropyl-1-(2-trifluoromethylphenyl)-1H-pyrazole-4-carbonyl]guanidine; [1-(2-trifluoromethyl-4-chlorophenyl)-5-cyclopropyl-1H-pyrazole-4-carbonyl]guanidine; and [5-cyclopropyl-1-(2-oxo-1,2-dihydro-quinolin-5-yl)-1H-pyrazole-4-carbonyl]guanidine.

This invention also provides methods of treating insulin resistance syndrome (IRS), type 2 diabetes, diabetic neuropathy, diabetic retinopathy, diabetic cardiomyopathy, diabetic microangiopathy, diabetic macroangiopathy, diabetic ischemia reperfusion injury, diabetic cardiac ischemia reperfusion injury, cataracts or foot ulcers in a mammal, which methods comprise administering to said mammal a combination of a first pharmaceutical agent, a prodrug or solvate of said first pharmaceutical agent or a pharmaceutically acceptable salt of said first pharmaceutical agent, said prodrug or said solvate of said first pharmacetical agent and a second pharmaceutical agent, a solvate of said second pharmaceutical agent or a pharmaceutically acceptable salt of said second pharmaceutical agent or said solvate of said second pharmaceutical agent, wherein:

said first pharmaceutical agent is a compound of Formula I as described above; and

said second pharmaceutical agent is a sulfonyl urea such as Glucotrol® (glipizide), Glucotrol XL® and glimepiride; a biguanide such as metformin (Glucovance®, Glucophage®, Mefformin GR™, ADX-155); a PPAR_{γ}/RXR agonist such as Avandia®, Actos®, darglitazone, troglitazone, JTT-501, MCC-555, MX6054, DRF2593, GI-262570, KRP-297 or LG100268; a PPAR-α/γ agonist such as NN-622, DRF-2725, BMS-298585, DRF-4158, KRP-297, DRF-2593, AZ-242 or NNC-61-0029; a PPAR-γ agonist such as CS-011; a partial PPAR-γ agonist such as FK-614 or MCC-555; an α-glucosidase inhibitor such as Precose® (acarbose) or Glyset® (miglitol); an anti-obesity agent such as Xenical® (orlistat) or Meridia®; a β-agonist such as TAK-667, AZ40140, CL-316243 or BRL 35135; an anti-diabetic agent such as ergoset or D-chiroinositol; a DPPIV inhibitor such as LAF-237, P-32/98,; an aldose reductase inhibitor; a glycogen phosphorylase inhibitor; a sorbitol dehydrogenase inhibitor; insulins such as Humulin R®, Humulin N®, Humulin 70/30®, Humalog®, Lantus®, Oralin and Macrulin; an insulin analog; Inhale Insulin; an insulin secretagogue such as repaglinide (Prandin®); a hepatic glucose output inhibitor such as meformin (above) and CS-917 (a fructose-1,6-bis-phosphatase inhibitor); T-1095; CRE-16257; a vanadate complex such as naglivan; vanadate complex; peroxyvanadate; an α2-agonist such as midaglizole; a fatty acid oxidation inhibitor such as clomoxir or etomoxir; a growth hormone secretagogue; a growth hormone mimetic; a lipid lowering agent such as benfluorex, Lipitor® (atorvastatin), Mevacor® (lovastatin), Zocor® (simvastatin), Pravachol® (pravastatin) or Lescol® (fluvastatin); amylin; Symlin™; an amylin antagonist such as pramlintide or AC-1 37; GLP-1 s such as exendin-4, AC2993, AC2993 LAR, LY-307161 and NN-2211; a lipoxygenase inhibitor such as masoprocal; an antilipolytic agent such as acipimox, a somatostatin analog such as BM-23014, seglitide or octreotide; a glucagon antagonist such as BAY 27-9955; an insulin sensitizer such as CRE-16336; an insulin signaling agonist, insulin mimetic or PTP1 B inhibitor such as PTP-112, ISIS-113715,L-783281, TER 17411 or TER 17529; an aP2 inhibitor; a SHIP2 inhibitor; a gluconeogenesis inhibitor such as GP3034; an insulin degrading enzyme inhibitor; a cGMP phosphodiesterase inhibitor such as Viagra® (sildenafil) or L686398; a cAMP phosphodiesterase inhibitor; a glucose transport stimulating agent such as BM-1 30795; a glycogen synthase kinase inhibitor such as lithium chloride, CT98014 or CT98023; a MTP inhibitor such as CP-651,802; a NPY inhibitor such as PD-160170, BW-383, BW-1229, CGP-71683A, NGD-95-1 or L-152804; an anorectic agent; a 5-HT2C receptor agonist; a 5-HT2C receptor mimetic; a 5HT receptor agonist; a 5-HT receptor mimetic; a CCKA agonist; a serotonin reuptake inhibitor such as Prozac® (fluoxetine) or Zoloft® (sertraline); a galanin receptor antagonist; a MCR-4 agonist such as HP-228; a leptinmimetic; leptin; a thyromimetic such as CGS-26214; a 11-13-hydroxysteroid dehydrogenase type-1 inhibitor; RU-486; urocortin; a glucocorticoid receptor antagonist; a urocortinmimetic; a CRF antagonist or a CRF binding protein. The combination is preferably administered in the form of a pharmaceutical composition additionally comprising a pharmaceutically acceptable vehicle, carrier or diluent.

The invention further provides methods of treating insulin resistance syndrome (IRS), type 2 diabetes, diabetic neuropathy, diabetic retinopathy, diabetic cardiomyopathy, diabetic microangiopathy, diabetic macroangiopathy, diabetic ischemia reperfusion injury, diabetic cardiac ischemia reperfusion injury, cataracts or foot ulcers in a mammal, which methods comprise administering to an individual in need thereof effective amounts of a first pharmaceutical agent, which is a compound of Formula I as set forth above, a prodrug or solvate thereof or a pharmaceutically acceptable salt of said compound, prodrug or solvate and two or more additional pharmaceutical agents selected from the second pharmaceutical agents set forth above. The combination is preferably adminstered in the form of a pharmaceutical composition additionally comprising a pharmaceutically acceptable vehicle, carrier or diluent.

The invention further provides methods of treating prophylactically an individual in whom Type 2 diabetes mellitus has not yet presented, but in whom there is an increased risk of developing such condition, which methods comprise administering to an individual in need thereof effective amounts of a first pharmaceutical agent, which is a compound of Formula I as set forth above, a prodrug or solvate thereof or a pharmaceutically acceptable salt of said compound, prodrug or solvate and a second pharmaceutical agent, wherein:

said first pharmaceutical agent is a compound of Formula I as described above; and

said second pharmaceutical agent is a(n) sulfonyl urea such as Glucotrol® (glipizide), Glucotrol XL® and glimepiride; a biguanide such as metformin (Glucovance®, Glucophage®, Metformin GR™, ADX-155); a PPAR_{γ}/RXR agonist such as Avandia®, Actos®, darglitazone, troglitazone, JTT-501, MCC-555, MX6054, DRF2593, GI-262570, KRP-297 or LG100268; a PPAR-α/γ agonist such as NN-622, DRF-2725, BMS-298585, DRF-4158, KRP-297, DRF-2593, AZ-242 or NNC-61-0029; a PPAR-γ agonist such as CS-011; a partial PPAR-γ agonist such as FK-614 or MCC-555; an α-glucosidase inhibitor such as Precose® (acarbose) or Glyset® (miglitol); an anti-obesity agent such as Xenical® (orlistat) or Meridiae; a β-agonist such as TAK-667, AZ40140, CL-316243 or BRL 35135; an anti-diabetic agent such as ergoset or D-chiroinositol; a DPPIV inhibitor such as LAF-237, P-32/98,; an aldose reductase inhibitor; a glycogen phosphorylase inhibitor; a sorbitol dehydrogenase inhibitor; insulins such as Humulin R®, Humulin N®, Humulin 70/30®, Humalog®, Lantus®, Oralin and Macrulin; an insulin analog; Inhale Insulin; an insulin secretagogue such as repaglinide (Prandin®); a hepatic glucose output inhibitor such as meformin (above) and CS-917 (a fructose-1,6-bis-phosphatase inhibitor); T-1095; CRE-16257; a vanadate complex such as naglivan; vanadate complex; peroxyvanadate; an α2-agonist such as midaglizole; a fatty acid oxidation inhibitor such as clomoxir or etomoxir; a growth hormone secretagogue; a growth hormone mimetic; a lipid lowering agent such as benfluorex, Lipitor® (atorvastatin), Mevacor® (lovastatin), Zocor® (simvastatin), Pravachol® (pravastatin) or Lescol® (fluvastatin); amylin; Symlin™; an amylin antagonist such as pramlintide or AC-1 37; GLP-1s such as exendin-4, AC2993, AC2993 LAR, LY-307161 and NN-2211; a lipoxygenase inhibitor such as masoprocal; an antilipolytic agent such as acipimox, a somatostatin analog such as BM-23014, seglitide or octreotide; a glucagon antagonist such as BAY 27-9955; an insulin sensitizer such as CRE-16336; an insulin signaling agonist, insulin mimetic or PTP1 B inhibitor such as PTP-112, ISIS-113715,L-783281, TER 17411 or TER 17529; an aP2 inhibitor; a SHIP2 inhibitor; a gluconeogenesis inhibitor such as GP3034; an insulin degrading enzyme inhibitor; a cGMP phosphodiesterase inhibitor such as Viagra® (sildenafil) or L686398; a cAMP phosphodiesterase inhibitor; a glucose transport stimulating agent such as BM-130795; a glycogen synthase kinase inhibitor such as lithium chloride, CT98014 or CT98023; a MTP inhibitor such as CP-651,802; a NPY inhibitor such as PD-160170, BW-383, BW-1229, CGP-71683A, NGD-95-1 or L-1 52804; an anorectic agent; a 5-HT2C receptor agonist; a 5-HT2C receptor mimetic; a 5HT receptor agonist; a 5-HT receptor mimetic; a CCKA agonist; a serotonin reuptake inhibitor such as Prozac® (fluoxetine) or Zoloft® (sertraline); a galanin receptor antagonist; a MCR-4 agonist such as HP-228; a leptinmimetic; leptin; a thyromimetic such as CGS-26214; a 11-β-hydroxysteroid dehydrogenase type-1 inhibitor; RU-486; urocortin; a glucocorticoid receptor antagonist; a urocortinmimetic; a CRF antagonist or a CRF binding protein. The combination is preferably adminstered in the form of a pharmaceutical composition additionally comprising a pharmaceutically acceptable vehicle, carrier or diluent.

The invention further provides methods of treating prophylactically an individual in whom insulin resistance syndrome (IRS), type 2 diabetes, diabetic neuropathy, diabetic retinopathy, diabetic cardiomyopathy, diabetic microangiopathy, diabetic macroangiopathy, diabetic ischemia reperfusion injury, diabetic cardiac ischemia reperfusion injury, cataracts or foot ulcers has not yet presented, but in whom there is an increased risk of developing such condition, which methods comprise administering to an individual in need thereof effective amounts of a first pharmaceutical agent, which is a compound of Formula I as set forth above, a prodrug or solvate thereof or a pharmaceutically acceptable salt of said compound, prodrug or solvate and two or more additional pharmaceutical agents selected from the second pharmaceutical agents set forth above. The combination is preferably adminstered in the form of a pharmaceutical composition additionally comprising a pharmaceutically acceptable vehicle, carrier or diluent.

The invention further provides methods of treating prophylactically an individual in whom Type 2 diabetes mellitus has not yet presented, but in whom there is an increased risk of developing such condition, which methods comprise administering to an individual in need thereof effective amounts of a first pharmaceutical agent, which is a compound of Formula I as set forth above, a prodrug or solvate thereof or a pharmaceutically acceptable salt of said compound, prodrug or solvate and two or more additional pharmaceutical agents selected from the second pharmaceutical agents set forth above. The combination is preferably adminstered in the form of a pharmaceutical composition additionally comprising a pharmaceutically acceptable vehicle, carrier or diluent.

This invention also provides kits comprising:

a) a first unit dosage form comprising a compound of Formula I, a prodrug or solvate thereof or a pharmaceutically acceptable salt of said compound of Formula I, said prodrug or said solvate and a pharmaceutically acceptable carrier, vehicle or diluent;

b) a second unit dosage form comprising:

a(n) sulfonyl urea, biguanide, PPAR_{γ} agonist, RXR agonist, α-glucosidase inhibitor, β-agonist, a DPPIV inhibitor, aldose reductase inhibitor, glycogen phosphorylase inhibitor, sorbitol dehydrogenase inhibitor, insulin, insulin analog, insulin secretagogue, vanadate complex, peroxyvanadate complex, α2-agonist, fatty acid oxidation inhibitor, growth hormone secretagogue, growth hormone mimetic, lipid lowering agent, amylin, amylin antagonist, lipoxygenase inhibitor, antilipolytic agent, somatostatin analog, glucagon antagonist, insulin signaling agonist, insulin mimetic, PTP1 B inhibitor, aP2 inhibitor, SHIP2 inhibitor, gluconeogenesis inhibitor, insulin degrading enzyme inhibitor, cAMP phosphodiesterase inhibitor, cGMP phosphodiesterase inhibitor, glucose transport stimulating agent, glycogen synthase kinase inhibitor, MTP inhibitor, NPY inhibitor, anorectic agent, 5-HT2C receptor agonist, 5-HT2C receptor mimetic, 5HT receptor agonist, 5-HT receptor mimetic, CCKA agonist, serotonin reuptake inhibitor, galanin receptor antagonist, MCR-4 agonist, leptinmimetic, thyromimetic, 11-β-hydroxysteroid dehydrogenase type-1 inhibitor, glucocorticoid receptor antagonist, urocortinmimetic, CRF antagonist or CRF binding protein; a solvate thereof or a pharmaceutically acceptable salt thereof or of said solvate and a pharmaceutically acceptable carrier, vehicle or diluent; and

c) a container.

Any individual representing a population having an increased risk of presenting with Type 2 diabetes mellitus may be prophylactically treated according to the methods of the instant invention. Accordingly, the methods of the invention are useful for preventing the transition to Type 2 diabetes mellitus of any disease state or condition associated with risk factors having the potential to cause or induce such transition. Examples of such risk factors may include, but are not limited to:

(i) risk factors associated with classification as an individual having insulin resistance and/or hyperinsulinemia;

(ii) risk factors based on an environmental or genetic Type 2 diabetes pre-disposing disease state or condition such as a family history of diabetes, especially in parents or siblings;

(iii) risk factors predicated on race and/or ethnicity, especially individual membership in a population comprising African-Americans, Hispanics, Native Americans, Asians, Pacific Islanders, and the like;

(iv) risk factors based on genetic mutations affecting β-cell function including defects on chromosome 12, gene HNF-1α (MODY3); defects on chromosome 7, gene glucokinase (MODY2); defects on chromosome 20, gene HNF-4α (MODYl); defects in mitochondrial DNA, and the like;

(v) risk factors based on genetic defects in insulin action including genetic mutations leading to Type A insulin resistance, acanthosis nigricans, leprechaunism, Rabson-Mendenhall syndrome, lipoatrophic diabetes or condition, or otherwise having a genetic mutation or mutations in the insulin receptor, IRS proteins, glucose transporters, PC-1, glucokinase, UCP-1, β3 adrenergic receptor gene, and the like;

(vi) risk factors based on presence of excess adipose tissue or clinically diagnosed obesity (i.e.≧20% excess of normal body weight, or BMI≧27 kg/m²), especially central obesity;

(vii) risk factors identified through clinical chemistries or diagnostic testing signifying a pre-diabetic state including impaired glucose tolerance (currently defined as impaired glucose response 2 hours following oral glucose load, i.e.≧140 mg/dl, but<200 mg/dl, with normal glucose fasting value), impaired fasting glucose (currently defined as fasting plasma glucose (FPG)≧110 mg/dl, but<126 mg/dl), or otherwise described as having hyperglycemia relative to normoglycemia;

(viii) risk factors related to physiologic and endocrine changes associated with growth, development, or aging such as classification as a menopausal, pubescent, or aged individual, especially an individual≧45 years of age;

(ix) risk factors related to diet or eating behaviors, including consumption of high fat or high carbohydrate diets, experiencing prolonged fasting or starvation, or risk factors associated with eating disorders, including having anorexia nervosa or bulemia, and the like;

(x) risk factors based on abnormal cardiovascular or blood lipid parameters, such as hypertension, i.e. blood pressure≧140/90 mmHg in adults, HDL cholesterol levels≦35 mg/dl and/or TG levels-≧250 mg/dl, or classification as having metabolic syndrome, i.e. Syndrome X;

(xi) risk factors based on reproductive status, such as pregnancy, or a history of gestational diabetes or macrosomia, i.e. the delivery of offspring having a birthweight of>9 lbs.;

(xii) risk factors attributable to muscle wasting due to aging, starvation, exposure to anti-gravity environments, paralysis resulting from spinal cord injury, and the like;

(xiii) risk factors associated with polycystic ovary syndrome;

(xiv) risk factors due to organ disease or dysfunction including liver cirrhosis, or renal disease;

(xv) risk factors due to conditions resulting in metabolic disturbances;

(xvi) risk factors due to endocrine disorders or endocrinopathies, such as hyperandrogenism, thyrotoxicosis, hyperthyroidism, insulinoma, glucagonoma, somatostatinoma, aldosteroma, Cushing's Syndrome, pheochromocytoma, acromegaly, hypercortisolemia, and the like;

(xvii) risk factors due to pathophysiologic states including infection (especially congenital rubella, cytomegalovirus, and the like), toxemia, uremia, sepsis, or trauma;

(xviii) risk factors due to immune-mediated disease such as “stiff man” syndrome, production of anti-insulin receptor antibodies, and the like;

(xix) risk factors due to drug or chemical exposure, including being treated with insulin-resistance-inducing or hyperglycemia-inducing agents including, for example, glucocorticoids, cytokines, α-interferon, thyroid hormone, TNFα, thiazides, certain estrogen-containing products, β-blockers, nicotinic acid, olanzapine and other serotonin receptor-targeted antipsychotics or antidepressants, vacor, diazoxide, dilantin, HIV protease inhibitors, and the like;

(xx) risk factors associated with having a genetic syndrome associated with diabetes including Down's Syndrome, Klinefelter's Syndrome, Wolfram's Syndrome, Freidreich's Syndrome, Huntington's chorea, Laurence-Moon-Biedl Syndrome, myotonic dystrophy, porphyria, Prader-Willi Syndrome, Alzheimer's Disease, and the like; and

(xxi) risk factors associated with the long-term detrimental effects caused by the administration of prolonged, elevated doses of insulin and/or the presence of ketoacidosis.

Generally preferred anti-diabetic agents useful as the second compound in the methods of this invention comprising administering a combination include, for example, D-chiroinositol; insulin and insulin analogs; GLP-1 (7-37)(insulinotropin) and GLP-1 (7-36)-NH₂; exendin, DPPIV inhibitors, α-glucosidase inhibitors; glitazones and/or insulin sensitizers; sulfonylureas and analogs thereof; biguanides; α2-antagonists and imidazolines; insulin secretagogues; aldose reductase inhibitors; fatty acid oxidation inhibitors; β-agonists; phosphodiesterase inhibitors; lipid-lowering agents; vanadate and vanadium complexes; amylin antagonists; glucagon antagonists; growth hormone secretagogues; gluconeogenesis inhibitors; somatostatin analogs; antilipolytic agents; lipoxygenase inhibitors; insulin signaling agonists; insulin mimetics; PTP1B inhibitors; aP2 inhibitors, SHIP2 inhibitors, insulin degrading enzyme inhibitors; glycogen synthase kinase inhibitors; and the like. Other non-NHE-1 inhibiting anti-diabetic agents, including the preferred agents set forth hereinbelow, are well known, or will be readily apparent in light of the instant disclosure, to one of ordinary skill in the art.

Preferred forms of insulin useful as the second pharmaceutical agent in the methods of the invention comprising administering a combination include, for example, inhaled insulin, or insulin analogs, for example, LysPro insulin. Preferred α-glucosidase inhibitors useful as the second pharmaceutical agent in the methods of the invention comprising administering a combination include those agents such as acarbose, voglibose, miglitol, emiglitate, camiglibose, MDL-25,637, and MDL-73,945. Preferred glitazones and/or insulin sensitizers useful as the second pharmaceutical agent in the methods of the invention comprising administering a combination include, for example, pioglitazone, rosiglitazone, JTT-501, MCC-555, and MX 6054. Preferred sulfonylureas and analogs thereof useful as the second pharmaceutical agent in the methods of the invention comprising administering a combination include, for example, chlorpropamide, glibenclamide, tolbutamide, tolazamide, acetohexamide, glipizide, glimepiride (Amaryl®), repaglinide (Prandin®), nateglinide (Starlix®) and meglitinide. Preferred biguanides useful as the second pharmaceutical agent in the methods of the invention comprising administering a combination include, for example, mefformin, phenformin, and buformin. Preferred α₂-antagonists and imidazolines useful as the second pharmaceutical agent in the methods of the invention comprising administering a combination include, for example, midaglizole, isaglidole, deriglidole, idazoxan, efaroxan, and fluparoxan. Preferred insulin secretagogues useful as the second pharmaceutical agent in the methods of the invention comprising administering a combination include, for example, linogliride, A-4166, exendin-4, and BTS-67582. Preferred aldose reductase inhibitors useful ias the second pharmaceutical agent in the methods of this invention comprising administering a combination include, for example, epalrestat, fidarestat, zopolrestat, zenarestat and tolrestat. Preferred fatty acid oxidation inhibitors useful as the second pharmaceutical agent in the methods of the invention comprising administering a combination include, for example, clomoxir and etomoxir. Preferred β-agonists useful as the second pharmaceutical agent in the methods of the invention comprising administering a combination include, for example, BRL-35135, BRL-37344, TAK-667, AZ 40140, and CL 316,243. Preferred phosphodiesterase inhibitors useful as the second pharmaceutical agent in the methods of the invention comprising administering a combination include, for example, L-386,398. Preferred lipid-lowering agents useful as the second pharmaceutical agent in the methods of the invention comprising administering a combination include, for example, benfluorex. Preferred vanadate and vanadium complexes useful as the second pharmaceutical agent in the methods of the invention comprising administering a combination include, for example, naglivan and peroxovandium complexes. Preferred gluconeogenesis inhibitors useful as the second pharmaceutical agent in the methods of the invention comprising administering a combination include, for example, glucose-6-phosphatase inhibitors, or GP 3034. Preferred antilipolytic agents useful as the second pharmaceutical agent in the methods of the invention comprising administering a combination include, for example, nicotinic acid, acipimox, and WAG 994. Preferred amylin antagonists useful as the second pharmaceutical agent in the methods of the invention comprising administering a combination include, for example, pramlintide and AC-137. Preferred glucagon antagonists useful as the second pharmaceutical agent in the methods of the invention comprising administering a combination include, for example, BAY 27-9955. Preferred lipoxygenase inhibitors useful as the second pharmaceutical agent in the methods of the invention comprising administering a combination include, for example, masoprocol. Preferred insulin signaling agonists useful as the second pharmaceutical agent in the methods of the invention comprising administering a combination include, for example, L-783281.

Generally preferred anti-obesity agents useful as the second pharmaceutical agent in the methods of this invention comprising administering a combination include, for example, β-adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (such as sibutramine), sympathiomimetic agents, serotoninergic agents (such as dexfenfluramine or fenfluramine), dopamine agonists (such as bromocriptine), melanocyte-stimulating hormone receptor agonists or mimetics, melanocyte-stimulating hormone analogs, melanin concentrating hormone antagonists, cannabinoid receptor antagonists, the OB protein (leptin), a leptin analog, galanin antagonists, lipase inhibitors (such as orlistat), anorectic agents, for example, bombesin agonists, Neuropeptide-Y antagonists, thyromimetic agents, dehydroepiandrosterones or analogs thereof, glucocorticoid receptor agonists or antagonists, 5HT2C receptor agonists, CB-1 receptor antagonists, 11-β-HSD inhibitors, orexin receptor antagonists, urocortin binding protein antagonists, glucagon-like peptide-1 receptor agonists, ciliary neurotrophic factors (such as Axokine), or human agouti-related protein (referred to hereinafter as AGRP) antagonists. Other anti-obesity agents, including the preferred agents set forth hereinbelow, are well known, or will be readily apparent in light of the instant disclosure, to one of ordinary skill in the art.

Particularly preferred anti-obesity agents useful as the second pharmaceutical agent in the methods of the invention comprising administering a combination include β-adrenergic receptor agonists, sibutramine, orlistat, fenfluramine, dexfenfluramine, bromocriptine, phentermine, ephedrine, leptin, phenylpropanolamine, and pseudoephedrine. Particularly preferred β-adrenergic receptor agonists include those substituted aminopyridines disclosed in commonly assigned PCT International Application Publication No. WO 96/35671, the disclosure of which is hereby incorporated by reference. Especially preferred β-adrenergic receptor agonists disclosed therein are selected from the group consisting of {4-[2-(2-[6-aminopyridin-3-yl]-2-(R)-hydroxyethylamino)ethoxy]phenyl}acetic acid, {4-[2-(2-[6-aminopyridin-3-yl]-2-(R)-hydroxyethylamino)ethoxy]phenyl}benzoic acid, {4-[2-(2-[6-aminopyridin-3-yl]-2-(R)-hydroxyethylamino)ethoxy]phenyl}propionic acid, and {4-[2-(2-[6-aminopyridin-3-yl]-2-(R)-hydroxyethylamino)ethoxy]phenoxy}acetic acid.

In another preferred embodiment of the methods comprising administering a combination, the second pharmaceutical agent is selected from LysPro insulin, GLP-1 (7-37) (insulinotropin), GLP-1 (7-36)-NH₂, chlorpropamide, glibenclamide, tolbutamide, tolazamide, acetohexamide, glypizide, glimepiride, repaglinide, meglitinide; metformin, phenformin, buformin, midaglizole, isaglidole, deriglidole, idazoxan, efaroxan, fluparoxan, linogliride, ciglitazone, pioglitazone, englitazone, troglitazone, darglitazone, rosiglitazone, clomoxir, etomoxir, acarbose, miglitol, emiglitate, voglibose, MDL-25,637, camiglibose, MDL-73,945, BRL 35135, BRL 37344, Ro 16-8714, ICI D7114, CL 316,243, L-386,398; benfluorex, fenfluramine, Naglivan®, acipimox, WAG 994, Symlin™, AC2993 and nateglinide.

In still another preferred embodiment of the methods comprising administering a combination, the second pharmaceutical agent is selected from insulin, sulfonylureas, biguanides, non-TZD PPAR agonists/antagonists and thiazolidinediones.

This invention is also directed to kits comprising:

a) a first unit dosage form comprising a compound of Formula I, a solvate thereof or a pharmaceutically acceptable salt of said compound of Formula I or said solvate and a pharmaceutically acceptable carrier, vehicle or diluent;

b) a second unit dosage form comprising:

a(n) sulfonyl urea, biguanide, PPAR, agonist, RXR agonist, α-glucosidase inhibitor, β-agonist, aldose reductase inhibitor, glycogen phosphorylase inhibitor, sorbitol dehydrogenase inhibitor, insulin, insulin analog, insulin secretagogue, vanadate complex, peroxyvanadate, α2-agonist, fatty acid oxidation inhibitor, growth hormone secretagogue, growth hormone mimetic, lipid lowering agent, amylin, amylin antagonist, lipoxygenase inhibitor, antilipolytic agent, somatostatin analog, glucagon antagonist, insulin signaling agonist, insulin mimetic, DPPIV inhibitor, CB-1 receptor antagonist, PTP1 B inhibitor, aP2 inhibitor, SHIP2 inhibitor, gluconeogenesis inhibitor, insulin degrading enzyme inhibitor, cAMP phosphodiesterase inhibitor, cGMP phosphodiesterase inhibitor, glucose transport stimulating agent, glycogen synthase kinase inhibitor, MTP inhibitor, NPY inhibitor, anorectic agent, 5-HT2C receptor agonist, 5-HT2C receptor mimetic, 5HT receptor agonist, 5-HT receptor mimetic, CCKA agonist, serotonin reuptake inhibitor, galanin receptor antagonist, MCR-4 agonist, leptinmimetic, thyromimetic, 11-β-hydroxysteroid dehydrogenase type-1 inhibitor, glucocorticoid receptor antagonist, urocortinmimetic, CRF antagonist or CRF binding protein; a solvate thereof or a pharmaceutically acceptable salt thereof or of said solvate and a pharmaceutically acceptable carrier, vehicle or diluent; and

c) a container.

The term “treating”, “treat” or “treatment” as used herein includes preventative (e.g., prophylactic) and palliative treatment.

By “pharmaceutically acceptable” it is meant the carrier, diluent, excipients, and/or salt must be compatible with the other ingredients of the formulation, and not deleterious to the recipient thereof.

Exemplary five to six membered aromatic rings optionally having one or two heteroatoms selected independently from oxygen, nitrogen and sulfur are phenyl, furyl, thienyl, pyrrolyl, oxazolyl, thiazolyl, imidazolyl, pyrazolyl, isoxazolyl, isothiazolyl, pyridinyl, pyridiazinyl, pyrimidinyl and pyrazinyl.

Exemplary partially saturated, fully saturated or fully unsaturated five to eight membered rings optionally having one to three heteroatoms selected independently from oxygen, sulfur and nitrogen are cyclopentyl, cyclohexyl, cycloheptyl, cyclooctyl and phenyl. Further exemplary five membered rings are furyl, thienyl, pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadizaolyl, 1,2,4-oxadiazolyl, 1,2,5-oxadiazolyl, 1,3,4-oxadiazolyl, 1,2,3-triazolyl, 1,2,4-trizaolyl, 1,3,4-thiadiazolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Further exemplary six membered rings are 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl and 1 ,4,2-oxadiazinyl.

Further exemplary seven membered rings are azepinyl, oxepinyl, thiepinyl and 1 ,2,4-diazepinyl.

Further exemplary eight membered rings are cyclooctyl, cyclooctenyl and cyclooctadienyl.

Exemplary bicyclic rings consisting of two fused partially saturated, fully saturated or fully unsaturated five and/or six membered rings, taken independently, optionally having one to four heteroatoms selected independently from nitrogen, sulfur and oxygen are indolizinyl, indolyl, isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazolyl, purinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

By alkylene is meant saturated hydrocarbon (straight chain or branched ) wherein a hydrogen atom is removed from each of the terminal carbons. Exemplary of such groups (assuming the designated length encompases the particular example) are methylene, ethylene, propylene, butylene, pentylene, hexylene, heptylene).

By halo is meant chloro, bromo, iodo, or fluoro.

By alkyl is meant straight chain saturated hydrocarbon or branched saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl.

By alkoxy is meant straight chain saturated alkyl or branched saturated alkyl bonded through an oxygen. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

As used herein the term mono-N- or di-N,N-(C₁-Cₓ)alkyl . . . refers to the (C₁-Cₓ)alkyl moiety taken independently when it is di-N,N-(C₁-Cₓ)alkyl . . . (x refers to integers).

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

The expression “prodrug” refers to compounds that are drug precursors which following administration, release the drug in vivo via some chemical or physiological process (e.g., a prodrug on being brought to the physiological pH or through enzyme action is converted to the desired drug form).

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. Where more than one basic moiety exists the expression includes multiple salts (e.g., di-salt). The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1 ,3-propanediol).

As used herein, the expressions “reaction-inert solvent” and “inert solvent” refers to a solvent or mixture of solvents which does not interact with starting materials, reagents, intermediates or products in a manner which adversely affects the yield of the desired product.

The term “treating”, “treat” or “treatment” as used herein includes preventative (e.g., prophylactic) and palliative treatment.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates of the compounds of this invention are also included.

DMF means N,N-dimethylformamide. DMSO means dimethyl sulfoxide. THF means tetrahydrofuran.

The subject invention also includes isotopically-labelled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I of this invention and prodrugs thereof can generally be prepared by carrying out the procedures disclosed in the Schemes and/or in the Examples below, by substituting a readily available isotopically labelled reagent for a non-isotopically labelled reagent.

Other features and advantages will be apparent from the specification and claims which describe the invention.

### DETAILED DESCRIPTION OF THE INVENTION

In general the compounds of this invention can be made by processes which include processes known in the chemical arts, particularly in light of the description contained in International Patent Application No. PCT/IB99/00206, which sets forth schemes, description and specific examples describing how to make the compounds used in the methods of the instant invention.

Some of the compounds used in the methods of this invention have asymmetric carbon atoms and therefore are enantiomers or diastereomers. Diasteromeric mixtures can be separated into their individual diastereomers on the basis of their physical chemical differences by methods known per se., for example, by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers and mixtures thereof are considered as part of this invention. Also, some of the compounds of this invention are atropisomers (e.g., substituted biaryls) and are considered as part of this invention.

Those skilled in the art will recognize that the compounds of Formula I can exist in several tautomeric forms. All such tautomeric forms can be used in the methods of this invention. For example, all of the tautomeric forms of the carbonylguanidine moiety of the compounds of Formula I are included in this invention. Also, for example, all enol-keto forms of the compounds of Formula I are included in this invention.

Some of the compounds used in the methods of this invention are acidic and they form a salt with a pharmaceutically acceptable cation. Some of the compounds used in the methods of this invention are basic and they form a salt with a pharmaceutically acceptable anion. All such salts, including di-salts are within the scope of this invention and they can be prepared by conventional methods. For example, they can be prepared simply by contacting the acidic and basic entities, in either an aqueous, non-aqueous or partially aqueous medium. The salts are recovered either by filtration, by precipitation with a non-solvent followed by filtration, by evaporation of the solvent, or, in the case of aqueous solutions, by lyophilization, as appropriate.

Metabolites, hydrates or solvates of the compounds of Formula I are within the scope of the compounds used in the methods of this invention.

In combination therapy treatment, both of the compounds of this invention and the other drug therapies are administered to mammals (e.g., humans, male or female) by conventional methods.

In general an effective dosage for the pharmacological combination compositions used in the methods of this invention, for example the diabetes prevention or treatment, Metabolic Syndrome prevention or treatment and/or diabetic neuropathy, diabetic cardiomyopathy, diabetic retinopathy, cataract, foot ulcer, diabetic macroangiopathy and/or diabetic microangiopathy activities of combinations containing the NHE-1 inhibitor compounds of this invention, is in the range of 0.005 to 50 mg/kg/day, preferably 0.01 to 25 mg/kg/day and most preferably 0.1 to 15 mg/kg/day.

The compounds of Formula I used in the methods of the present invention inhibit the sodium/proton (Na+/H+) exchange transport system and hence are useful as a therapeutic or prophylactic agent for diseases caused or aggravated by the acceleration of the sodium/proton (Na+/H+) exchange transport system, for example, type 2 diabetes, IRS, diabetic neuropathy, diabetic cardiomyopathy, diabetic retinopathy, diabetic microangiopathy, diabetic macroangiopathy, foot ulcers and cataracts.

The utility of the compounds of Formula I used in the methods of the present invention as medical agents in the treatment of type 2 diabetes, IRS, diabetic neuropathy, diabetic cardiomyopathy, diabetic retinopathy, cataracts, foot ulcers, diabetic macroangiopathy and diabetic microangiopathy, such as are detailed herein in mammals (e.g. humans) is demonstrated by the activity of the compounds of this invention in conventional preclinical NHE-1 Inhibitory Activity assays. Such assays also provide a means whereby the activities of the compounds of this invention can be compared with the activities of other known compounds. The results of these comparisons are useful for determining dosage levels in mammals, including humans, for the treatment of such diseases.

Measurement of Human NHE-1 Inhibitory Activity

Methodologies for measurement of human NHE-1 activity and inhibitor potency are based on those published by Watson et al., Am. J. Physiol., 24:G229-G238, 1991), where NHE-mediated recovery of intracellular pH is measured following intracellular acidification. Thus, fibroblasts stably expressing human NHE-1 (Counillon, L. et al., Mol. Pharmacol., 44:1041-1045 (1993) are plated onto collagen coated 96 well plates (50,000/well) and grown to confluence in growth media (DMEM high glucose, 10% fetal bovine serum, 50 μ/ml penicillin and streptomycin). Confluent plates are incubated for 30 min at 37° C. with the pH sensitive fluorescent probe BCECF (5 μM; Molecular Probes, Eugene, Oreg.). BCECF loaded cells are incubated for 30 min at 37° C. in acid loading media (70 mM choline chloride, 50 mM NHCl₄, 5 mM KCl, 1 mM MgCl₂, 1.8 mM CaCl₂, 5 mM glucose, 10 mM HEPES, pH 7.5), and then placed in a Fluorescent Imaging Plate Reader (Molecular Devices, CA). BCECF fluorescence is monitored using excitation and emission wavelengths of 485 nM and 525 nM, respectively. Intracellular acidification is initiated via rapid replacement of acid loading media with recovery media (120 mM NaCl, 5 mM KCl, 1 mM MgCl₂, 1.8 mM CaCl₂, 5 mM glucose, 10 mM HEPES, pH 7.5)±test compound, and NHE-mediated recovery of intracellular pH is monitored as the subsequent time-dependent increase BCECF fluorescence. The potency of human NHE-1 inhibitors is calculated as the concentration that reduces recovery of intracellular pH by 50% (IC₅₀). Under these conditions reference NHE inhibitors amiloride and HOE-642 had IC₅₀ values for human NHE-1 of 50 μM and 0.5 μM, respectively.

Administration of the compounds used in the methods of this invention can be via any method which delivers said compound preferentially to the desired tissue. These methods include oral routes, parenteral, intraduodenal routes, etc. Generally, the compounds used in the methods of the present invention are administered in single (e.g., once daily) or multiple doses or via constant infusion.

The ability of an NHE-1 inhibitor or a combination of an NHE-1 inhibitor and a second pharmaceutical agent as defined above to treat prophylactically an individual in whom Type 2 diabetes mellitus has not yet presented, but in whom there is an increased risk of developing such condition, may be demonstrated according to the following exemplary, non-limiting protocols.

The experimetal protocol described by Sreenan, et al., Am. J. Physiol., 271, E742-747 (1996), may be employed to evaluate the NHE-1 inhibitor or a combination of the NHE-1 inhibitor and a second pharmaceutical agent for the ability to prevent or delay the onset of diabetes in the prone obese Zucker diabetic fatty rat (Charles River Labs, Wilmington, MA and Genetic Models Inc.; Indianapolis, Ind.), or for the delay or prevention of the onset of insulin resistance or impaired glucose toleration in the prone obese Zucker fatty rat.

Rats six weeks of age may be initiated on a daily regimen of treatment employing the NHE-1 inhibitor or a combination of the NHE-1 inhibitor and a second pharmaceutical agent (p.o. by gavage or in the chow), while being maintained on a standard rodent diet (Purina 5008; W.F. Fisher & Son, Inc., Bound Brook, N.J.). After six weeks, the rats are fasted overnight, and blood samples are taken for determination of serum glucose, insulin, triglyceride, and free fatty acid concentrations. The results for the treated rats are compared against the untreated rats, and also against the lean littermates, which are considered normal. A reduction in serum glucose, insulin, triglyceride, and/or free fatty acid levels in the treated group compared to the untreated group indicates delay or prevention of the onset of diabetes or insulin resistance attributable to the NHE-1 inhibitor or a combination of the NHE-1 inhibitor and a second pharmaceutical agent. The animals can also be administered a glucose tolerance test or examined in the postprandial (fed) state after the six week treatment period. A reduction in serum glucose or insulin levels during the glucose tolerance test period or in the fed state by the treated group compared to the untreated group also indicates that the NHE-1 inhibitor or a combination of the NHE-1 inhibitor and a second pharmaceutical agent delayed or prevented the onset of diabetes, insulin resistance, and/or impaired glucose tolerance.

According to the methods described by Thomas, et al., Biochem. Pharm., 56, 1145-1150 (1998), NHE-1 inhibitor or a combination of the NHE-1 inhibitor and a second pharmaceutical agent can also be tested for delay or prevention of the onset of insulin resistance in dexamethasone-induced hyperglycemic and insulin resistant mice.

C57BL6 (+/+) mice (Jackson Laboratory; Bar Harbor, Me.) 15 weeks of age may be treated with dexamethasone at 2.5 mg/kg/day plus the NHE-1 inhibitor or a combination of the NHE-1 inhibitor and a second pharmaceutical agent (p.o. by gavage) or vehicle (untreated) for ten days. At the end of the ten-day period, blood samples are taken for plasma glucose and insulin determination in the fed state. The animals are fasted for 12 hr., and subjected to an insulin tolerance test, comprising blood sampling at 10, 20, and 40 min. after i.p. administration of 0.5 U/kg insulin, for calculation of plasma glucose disappearance. Alternatively, a glucose tolerance test may be administered to the fasted animals after the ten-day treatment period. A reduction in plasma glucose or insulin levels in the fed state, a greater plasma glucose disappearance during the insulin tolerance test, and/or lower glucose or insulin levels during the glucose tolerance test by the NHE-1 inhibitor or a combination of the NHE-1 inhibitor and a second pharmaceutical agent plus dexamethasone-treated group compared to the dexamethasone control group indicates that the NHE-1 inhibitor or a combination of the NHE-1 inhibitor and a second pharmaceutical agent prevented or delayed the onset of insulin resistance, hyperglycemia, and impaired glucose tolerance by the dexamethasone treatment.

Further, according to the method described by Davidson, et al., Am. J. Physiol., 264, El 8-23, (1993), NHE-1 inhibitor or a combination of the NHE-1 inhibitor and a second pharmaceutical agent may also be tested for the ability to delay or prevent the onset of insulin resistance induced by a cafeteria diet treatment of rats.

Male Sprague-Dawley rats (Charles Rivers Labs; Wilmington, MA) weighing 200 g are fed a cafeteria diet consisting of braunschweiger liver sausage, assorted candy bars, cheeses, cookies, corn chips, granola bars, marshmallows, peanut butter, Twinkies, and sweetened condensed milk plus the NHE-1 inhibitor or a combination of the NHE-1 inhibitor and a second pharmaceutical agent (p.o. by gavage or in the diet) or vehicle (untreated) for seven to forty-two days. Alternatively, a high sucrose or high fat diet may be used; such diets are well known to those skilled in the art. At the end of the seven to forty-two day period, blood samples are taken for plasma glucose and insulin determination in the fed state. Body weight and adipose depot weight are also determined. A reduction in plasma glucose, lipid or insulin levels in the fed state, and/or lower glucose or insulin levels during the glucose tolerance test, and/or reduced weight gain or obesity (adipose depot weight) by the NHE-1 inhibitor or a combination of the NHE-1 inhibitor and a second pharmaceutical agent plus cafeteria diet-treated group compared to the cafeteria-fed control group will indicate that the NHE-1 inhibitor or a combination of the NHE-1 inhibitor and a second pharmaceutical agent delayed or prevented the onset of insulin resistance, hyperglycemia, and impaired glucose tolerance induced by the cafeteria diet treatment.

The utility of the compounds of the present invention as medical agents in the treatment or prevention of diseases (such as are detailed herein) in animals, particularly mammals (e.g. humans) is demonstrated by the activity of the compounds of this invention in conventional assays and the in vitro and in vivo assays described below. Such assays also provide a means whereby the activities of the compounds of this invention can be compared with the activities of other known compounds. The results of these comparisons are useful for determining dosage levels in animals, particularly mammals, including humans, for the treatment of such diseases.

The compounds of this invention are readily adapted to clinical use as antidiabetic agents. The hypoglycemic activity of the compounds of this invention can be determined by the amount of test compound that reduces glucose levels relative to a vehicle without test compound in male ob/ob mice. The test also allows the determination of an approximate minimal effective dose (MED) value for the in vivo reduction of plasma glucose concentration in such mice for such test compounds.

Since the concentration of glucose in blood is closely related to the development of diabetic disorders, the compounds of the present invention, by virtue of their hypoglycemic action, prevent, arrest and/or regress diabetic disorders.

Five to eight week old male C57BLU6J-ob/ob mice (obtained from Jackson Laboratory, Bar Harbor, Me.) are housed five per cage under standard animal care practices. After a one week acclimation period, the animals are weighed and 25 microliters of blood are collected from the retro-orbital sinus prior to any treatment. The blood sample is immediately diluted 1:5 with saline containing 0.025% sodium heparin, and held on ice for metabolite analysis. Animals are assigned to treatment groups so that each group has a similar mean for plasma glucose concentration. After group assignment, animals are dosed orally each day for four days with the vehicle consisting of either: (1) 0.25% w/v methyl cellulose in water without pH adjustment; or (2) 0.1% Pluronic® P105 Block Copolymer Surfactant (BASF Corporation, Parsippany, N.J.) in 0.1% saline without pH adjustment. On day 5, the animals are weighed again and then dosed orally with a test compound or the vehicle alone. All compounds are administered in vehicle consisting of either: (1) 0.25% w/v methyl cellulose in water; or 3) neat PEG 400 without pH adjustment; (2) 10% DMSO/O.1% Pluronic® in 0.1% saline without pH adjustment; or 3) neat PEG 400 without pH adjustment. The animals are then bled from the retro-orbital sinus three hours later for determination of blood metabolite levels. The freshly collected samples are centrifuged for two minutes at 10,000×g at room temperature. The supernatant is analyzed for glucose, for example, by the Abbott VP™ (Abbott Laboratories, Diagnostics Division, Irving, Tex.) and VP Super System® Autoanalyzer (Abbott Laboratories, Irving, Tex.), or by the Abbott Spectrum CCX™ (Abbott Laboratories, Irving, Tex.) using the A-Gent™ Glucose-UV Test reagent system (Abbott Laboratories, Irving, Tex.) (a modification of the method of Richterich and Dauwalder, Schweizerische Medizinische Wochenschrift, 101: 860 (1971)) (hexokinase method) using a 100 mg/dl standard. Plasma glucose is then calculated by the equation: Plasma glucose (mg/dl)=Sample value×8.14

where 8.14 is the dilution factor, adjusted for plasma hematocrit (assuming the hematocrit is 44%).

The animals dosed with vehicle maintain substantially unchanged hyperglycemic glucose levels (e.g., greater than or equal to 250 mg/dl), animals treated with compounds having hypoglycemic activity at suitable doses have significantly depressed glucose levels. Hypoglycemic activity of the test compounds is determined by statistical analysis (unpaired t-test) of the mean plasma glucose concentration between the test compound group and vehicle-treated group on day 5. The above assay carried out with a range of doses of a test compound allows the determination of an approximate minimal effective dose (MED) value for the in vivo reduction of plasma glucose concentration.

The compounds of the present invention are readily adapted to clinical use as hyperinsulinemia reversing agents, triglyceride lowering agents and hypocholesterolemic agents. Such activity can be determined by the amount of test compound that reduces insulin, triglycerides or cholesterol levels relative to a control vehicle without test compound in male ob/ob mice.

Since the concentration of cholesterol in blood is closely related to the development of cardiovascular, cerebral vascular or peripheral vascular disorders, the compounds of this invention, by virtue of their hypocholesterolemic action, prevent, arrest and/or regress atherosclerosis.

Since the concentration of insulin in blood is related to the promotion of vascular cell growth and increased renal sodium retention, (in addition to the other actions, e.g., promotion of glucose utilization) and these functions are known causes of hypertension, the compounds of this invention, by virtue of their hypoinsulinemic action, prevent, arrest and/or regress hypertension.

Since the concentration of triglycerides in blood contributes to the overall levels of blood lipids, the compounds of this invention, by virtue of their triglyceride lowering and/or free fatty acid lowering activity prevent, arrest and/or regress hyperlipidemia.

Free fatty acids contribute to the overall level of blood lipids and independently have been negatively correlated with insulin sensitivity in a variety of physiologic and pathologic states.

Five to eight week old male C57BL/6J-ob/ob mice (obtained from Jackson Laboratory, Bar Harbor, Me.) are housed five per cage under standard animal care practices and fed standard rodent diet ad libitum. After a one week acclimation period, the animals are weighed and 25 microliters of blood are collected from the retro-orbital sinus prior to any treatment. The blood sample is immediately diluted 1:5 with saline containing 0.025% sodium heparin, and held on ice for plasma glucose analysis. Animals are assigned to treatment groups so that each group has a similar mean for plasma glucose concentration. The compound to be tested is administered by oral gavage as an about 0.02% to 2.0% solution (weight/volume (w/v)) in either (1) 10% DMSO/0.1% Pluronic® P105 Block Copolymer Surfactant (BASF Corporation, Parsippany, N.J.) in 0.1% saline without pH adjustment or (2) 0.25% w/v methylcellulose in water without pH adjustment. Alternatively, the compound to be tested can be administered by oral gavage dissolved in or in suspension in neat PEG 400. Single daily dosing (s.i.d.), twice daily dosing (b.i.d.) or other dosing regimen is maintained for 1 to, for example, 15 days. Control mice receive the 10% DMSO/0.1% Pluronic® P105 in 0.1 % saline without pH adjustment or the 0.25% w/v methylcellulose in water without pH adjustment, or the neat PEG 400 without pH adjustment.

Three hours after the last dose is administered, the animals are sacrificed by decapitation and trunk blood is collected into 0.5 ml serum separator tubes containing 3.6 mg of a 1:1 weight/weight sodium fluoride: potassium oxalate mixture. The freshly collected samples are centrifuged for two minutes at 10,000×g at room temperature, and the serum supernatant is transferred and diluted 1:1 volume/volume with a 1TIU/ml aprotinin solution in 0.1% saline without pH adjustment.

The diluted serum samples are then stored at −80° C. until analysis. The thawed, diluted serum samples are analyzed for insulin, triglycerides, free fatty acids and cholesterol levels. Serum insulin concentration is determined using Equate® RIA INSULIN kits (double antibody method; as specified by the manufacturer) available from Binax, South Portland, Me. The inter assay coefficient of variation is ≦10%. Serum triglycerides are determined using the Abbott VP™ and VP Super System® Autoanalyzer (Abbott Laboratories, Irving, Tex.), or the Abbott Spectrum CCX™ (Abbott Laboratories, Irving, Tex.) using the A-Gent™ Triglycerides Test reagent system (Abbott Laboratories, Diagnostics Division,lrving, Tex.) (lipase-coupled enzyme method; a modification of the method of Sampson, et al., Clinical Chemistry 21: 1983 (1975)). Serum total cholesterol levels are determined using the Abbott VP™ and VP Super System® Autoanalyzer (Abbott Laboratories, Irving, Tex.), and A-Gent™ Cholesterol Test reagent system (cholesterol esterase-coupled enzyme method; a modification of the method of Allain, et al. Clinical Chemistry 20: 470 (1974)) using 100 and 300 mg/dl standards. Serum free fatty acid concentration is determined utilizing a kit from Amano International Enzyme Co., Inc., as adapted for use with the Abbott VP™ and VP Super System® Autoanalyzer (Abbott Laboratories, Irving, Tex.), or the Abbott Spectrum CCX™ (Abbott Laboratories, Irving, Tex.). Serum insulin, triglycerides, free fatty acids and total cholesterol levels are then calculated by the equations,

Serum insulin (μU/ml)=Sample value×2

Serum triglycerides (mg/dl)=Sample value×2

Serum total cholesterol (mg/dl)=Sample value×2

Serum free fatty acid (μEq/l)=Sample value×2

where 2 is the dilution factor.

The animals dosed with vehicle maintain substantially unchanged, elevated serum insulin (e.g., 275 μU/ml), serum triglycerides (e.g., 235 mg/dl), serum free fatty acid (1500 mEq/ml) and serum total cholesterol (e.g., 190 mg/dl) levels, while animals treated with compounds of the present invention generally display reduced serum insulin, triglycerides, free fatty acid and total cholesterol levels. The serum insulin, triglycerides, free fatty acid and total cholesterol lowering activity of the test compounds are determined by statistical analysis (unpaired t-test) of the mean serum insulin, triglycerides, or total cholesterol concentration between the test compound group and the vehicle-treated control group.

Generally, the compounds used in the methods of this invention are administered orally, or parenterally (e.g., intravenous, intramuscular, subcutaneous or intramedullary). Topical administration may also be indicated, for example, where the patient is suffering from gastrointestinal disorders or whenever the medication is best applied to the surface of a tissue or organ as determined by the attending physician.

The amount and timing of compounds administered will, of course, be dependent on the subject being treated, on the severity of the affliction, on the manner of administration and on the judgement of the prescribing physician. Thus, because of patient to patient variability, the dosages given below are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases (e.g., cardiovascular disease).

In the methods of this invention, an amount of the compounds used in the methods of this invention is used that is effective for treatment of type 2 diabetes, IRS, diabetic neuropathy, diabetic cardiomyopathy, diabetic retinopathy, diabetic microangiopathy, diabetic macroangiopathy, foot ulcers and/or cataracts. A preferred dosage is about 0.001 to 100 mg/kg/day of the compound of this invention. An especially preferred dosage is about 0.01 to 50 mg/kg/day of the compound of this invention.

The compounds used in the methods of the present invention are generally administered in the form of a pharmaceutical composition comprising at least one of the compounds of this invention together with a pharmaceutically acceptable vehicle or diluent. Thus, the compounds used in the methods of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions, for example, in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

Pharmaceutical compositions used in the methods of this invention may contain for example 0.0001 %-95% of a compound of Formula I above. In any event, the composition or formulation to be administered will contain a quantity of a compound of Formula I in an amount effective to treat diabetes or one or more diabetic complications of the subject being treated, as required.

When a combination of two compounds is used in a method of this invention, the two different compounds used can be co-administered simultaneously or sequentially in any order, or as a single pharmaceutical composition comprising a compound of Formula I a second pharmaceutical agent as set forth above.

Since the present invention has an aspect that relates to the treatment of the diabetes and/or one or more diabetic complications with a combination of active ingredients which may be administered separately, the invention also relates to combining separate pharmaceutical compositions in kit form. The kit comprises two separate pharmaceutical compositions: a compound of Formula I or a pharmaceutically acceptable salt of such compound and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules or vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLE 1

The antidiabetic effect of NHE-1 inhibitors [5-cyclopropyl-1-(quinolin-5-yl)-1H-pyrazole-4-carbonyl]guanidine and [1-(2-trifluoromethyl-4-chlorophenyl)-5-cyclopropyl-1H-pyrazole-4-carbonyl]-guanidine were examined in diabetic ob/ob mice (as described above). Values reported below are as the mean ±SD for number of animals indicated. Statistical analyses were performed by Student t-test. Study 1: ob/ob mice (n=10) were administered [5-cyclopropyl-1-(quinolin-5-yl)-1H-pyrazole-4-carbonyl]guanidine at a dose of 20 mg/kg qd for five days. 3 hours after the dose was administered on the fifth day, a blood sample was collected and analyzed for plasma glucose and insulin concentration. The results were compared to a vehicle treated group of ob/ob mice (n=10) carried in parallel the same experiment.

Results:

| | Day 5, 3h post-dose | |
|---|---|---|
| | plasma glucose | Day 5, 3h post-dose plasma |
| Group | concentration, mg/dl | insulin concentration, ug/l |
| Vehicle Control | 482 ± 139 | 57.7 ± 24.5 |
| [5-cyclopropyl-1- | 371 ± 90 | 20.5 ± 10.3 |
| (quinolin-5-yl)-1H- | (p = 0.0498) | (p = 0.000331) |
| pyrazole-4-carbonyl] | | |
| guanidine-treated | | |

Note-Plasma glucose concentration for groups above were similar when measured on Day 1 before the initiation of treatment: Vehicle control = 371 ± 38 mg/dl, [5-cyclopropyl-1-(quinolin-5-yl)-1H-pyrazole-4-carbonyl]guanidine-treated = 371 ± 38 mg/dl.

Conclusion: The NHE-1 Inhibitor [5-cyclopropyl-1-(quinolin-5-yl)-1H-pyrazole-4-carbonyl]guanidine significantly reduced plasma glucose and plasma insulin concentration in diabetic ob/ob mice compared to the vehicle control group.

Study 2: ob/ob mice (n=10) were administered [1-(2-trifluoromethyl-4-chlorophenyl)-5-cyclopropyl-1H-pyrazole-4-carbonyl]-guanidine at a dose of 20 mg/kg bid for four days. 3 hours after an AM dose was administered on the fifth day, a blood sample was collected and analyzed for plasma glucose and insulin concentration. The results were compared to a vehicle treated group of ob/ob mice (n=10) carried in parallel the same experiment.

Results:

| | Day 5, 3h post-dose | Day 5, 3h post-dose |
|---|---|---|
| | plasma glucose | plasma insulin |
| Group | concentration, mg/dl | concentration, ug/l |
| Vehicle Control | 322 ± 72 | 10.0 ± 6.8 |
| [1-(2-trifluoromethyl-4- | 195 ± 50 | 11.1 ± 9.0 |
| chlorophenyl)-5-cyclopropyl- | (p = 0.0003) | |
| 1H-pyrazole-4-carbonyl]- | | |
| guanidine-treated | | |

Note-Plasma glucose concentration for groups above were similar (p > 0.05) when measured on Day 1 before the initiation of treatment: Vehicle control = 373 ± 57 mg/d, [1-(2-trifluoromethyl-4-chlorophenyl)-5-cyclopropyl-1H-pyrazole-4-carbonyl]-guanidine-treated = 360 ± 61 mg/dl.

Conclusion: The NHE-1 Inhibitor [1-(2-trifluoromethyl-4-chlorophenyl)-5-cyclopropyl-1H-pyrazole-4-carbonyl]-guanidine significantly reduced plasma glucose concentration in diabetic ob/ob mice compared to the vehicle control group.

Study 3: ob/ob mice (n=15) were administered [1-(2-trifluoromethyl-4-chlorophenyl)-5-cyclopropyl-1H-pyrazole-4-carbonyl]-guanidine at a dose of 20 mg/kg bid for seven days. 3 hours after an AM dose was administered on the eigth day, a blood sample was collected and analyzed for plasma glucose, triglyceride, and insulin concentration. The results were compared to a vehicle treated group of ob/ob mice (n=15) carried in parallel the same experiment.

Results:

| | Day 8, 3h post- | | Day 8, 3h post- |
|---|---|---|---|
| | dose plasma | | dose plasma |
| | glucose | Day 8, 3h post-dose | triglyceride |
| | concentration, | plasma insulin | concentration, |
| Group | mg/dl | concentration, ug/l | mg/dl |
| Vehicle Control | 397 ± 134 | 26 ± 8 | 167 ± 44 |
| [1-(2-trifluoro- | 204 ± 69 | 16 ± 59 | 107 ± 24 |
| methyl-4-chloro- | (p = 0.0001) | (p = 0.005) | (p = 0.0002) |
| phenyl)-5-cyclo- | | | |
| propyl-1H-pyra- | | | |
| zole-4-carbon- | | | |
| yl]-guanidine- | | | |
| treated | | | |

Note-Plasma glucose concentration for groups above were similar (p > 0.05) when measured on Day 1 before the initiation of treatment: Vehicle control = 466 ± 64 mg/dl, [1-(2-trifluoromethyl-4-chlorophenyl)-5-cyclopropyl-1H-pyrazole-4-carbonyl]-guanidine-treated = 459 ± 102 mg/dl.

Conclusion: The NHE1 Inhibitor [1-(2-trifluoromethyl-4-chlorophenyl)-5-cyclopropyl-1H-pyrazole-4-carbonyl]-guanidine significantly reduced plasma glucose concentration, plasma insulin concentration and plasma triglyceride concentration in diabetic ob/ob mice compared to the vehicle control group.

1. A method of treating insulin resistance syndrome (IRS) in a mammal comprising administering to'said mammal a compound of Formula I: 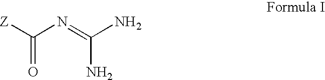
a prodrug or solvate thereof or a pharmaceutically acceptable salt of said compound, prodrug or solvate, wherein
Z is carbon connected and is a five-membered, diaza, diunsaturated ring having two contiguous nitrogens, said ring optionally mono-, di-, or tri-substituted with up to three substituents independently selected from R¹, R² and R³; or
Z is carbon connected and is a five-membered, triaza, diunsaturated ring, said ring optionally mono- or di-substituted with up to two substituents independently selected from R⁴ and R⁵;
wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines.

2. A method of treating type 2 diabetes in a mammal comprising administering to said mammal a compound of Formula I: 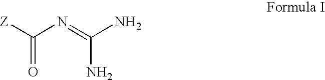
a prodrug or solvate thereof or a pharmaceutically acceptable salt of said compound, prodrug or solvate, wherein
Z is carbon connected and is a five-membered, diaza, diunsaturated ring having two contiguous nitrogens, said ring optionally mono-, di-, or tri-substituted with up to three substituents independently selected from R¹, R² and R³; or
Z is carbon connected and is a five-membered, triaza, diunsaturated ring, said ring optionally mono- or di-substituted with up to two substituents independently selected from R⁴ and R⁵;
wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines.

3. A method of treating diabetic neuropathy, diabetic retinopathy, diabetic cardiomyopathy, cataracts, foot ulcers, diabetic ischemia reperfusion injury, diabetic cardiac ischemia reperfusion injury, diabetic microangiopathy or diabetic macroangiopathy in a mammal comprising administering to said mammal a compound of Formula I: 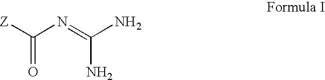
a prodrug or solvate thereof or a pharmaceutically acceptable salt of said compound, prodrug or solvate, wherein
Z is carbon connected and is a five-membered, diaza, diunsaturated ring having two contiguous nitrogens, said ring optionally mono-, di-, or tri-substituted with up to three substituents independently selected from R¹, R² and R³; or
Z is carbon connected and is a five-membered, triaza, diunsaturated ring, said ring optionally mono- or di-substituted with up to two substituents independently selected from R⁴ and R⁵;
wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C, -C₄)alkoxy, (C, -C₄)alkoxy(Cl -C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines.

4. A method of claim 3 wherein diabetic neuroapathy is treated.

5. A method of claim 3 wherein diabetic retinopathy is treated.

6. A method of claim 3 wherein diabetic cardiomyopathy is treated.

7. A method of claim 3 wherein cataracts are treated.

8. A method of claim 3 wherein diabetic microangiopathy is treated.

9. A method of claim 3 wherein diabetic macroangiopathy is treated.

10. A method of claim 3 wherein diabetic ischemia reperfusion injury is treated.

11. A method of claim 3 wherein diabetic cardiac ischemia reperfusion injury is treated.

12. A method of treating prophylactically an individual in whom Type 2 diabetes has not yet presented, but in whom there is an increased risk of developing such a condition comprising administering to said individual a compound of Formula I: 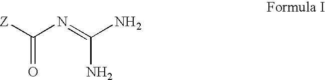
a prodrug or solvate thereof or a pharmaceutically acceptable salt of said compound, prodrug or solvate, wherein
Z is carbon connected and is a five-membered, diaza, diunsaturated ring having two contiguous nitrogens, said ring optionally mono-, di-, or tri-substituted with up to three substituents independently selected from R¹, R² and R³; or
Z is carbon connected and is a five-membered, triaza, diunsaturated ring, said ring optionally mono- or di-substituted with up to two substituents independently selected from R⁴ and R⁵;
wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines.

13. A method of any one of claims 1-12 wherein in said compound 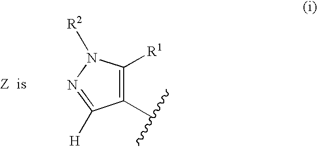
wherein R¹ is (C₃-C₇)cycloalkyl, phenyl or phenyl(C₁-C₄)alkyl, said (C₃-C₇)cycloalkyl optionally substituted with from one to three fluorines, said R¹ substituent optionally mono- or di-substituted independently with (C₁-C₄)alkoxy, (C₁-C₄)alkylthio, (C₁-C₄)alkylsulfinyl or (C₁-C₄)alkylsulfonyl; and R² is (C₁-C₄)alkyl, (C₃-C₄)cycloalkyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N7(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; or 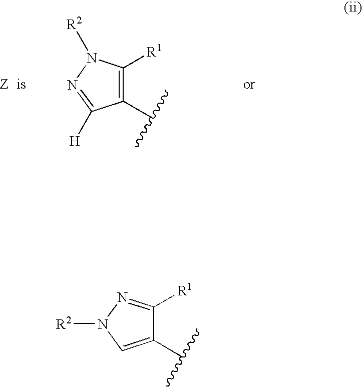
wherein R¹ is (C₁-C₄)alkyl, (C₃-C₇)cycloalkyl, phenyl or phenyl(C₁-C₄)alkyl, said (C₁-C₄)alkyl optionally substituted with from one to nine fluorines, said R¹ substituent optionally mono- or di-substituted independently with (C₁-C₄)alkoxy, (C₁-C₄)alkylthio, (C₁-C₄)alkylsulfinyl or (C₁-C₄)alkylsulfonyl; and
R² is a five to six membered nonaromatic heterocyclic ring having one to two heteroatoms selected independently from nitrogen, sulfur and oxygen or R² is unsubstituted (C₁-C₄)alkyl or unsubstituted (C₃-C₇)cycloalkyl; or R² is phenyl(C₁-C₄)alkyl, or a bicyclic ring consisting of two fused five and/or six membered partially saturated, fully saturated or fully unsaturated rings taken independently having one to four heteroatoms selected independently from nitrogen, sulfur and oxygen, said R² substituents optionally substituted on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines, or 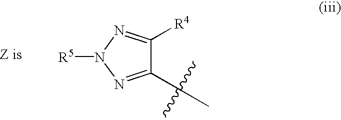
wherein R⁴ is (C₁-C₄)alkyl, (C₃-C₇)cycloalkyl, phenyl or phenyl(C₁-C₄)alkyl, said (C₁-C₄)alkyl optionally substituted with from one to nine fluorines, said R⁴ substituent optionally mono- or di-substituted independently with (C₁-C₄)alkoxy, (C₁-C₄)alkylthio, (C₁-C₄)alkylsulfinyl or (C₁-C₄)alkylsulfonyl; and
R⁵ is a five to six membered nonaromatic heterocyclic ring having one to two heteroatoms selected independently from nitrogen, sulfur and oxygen or R⁵ is unsubstituted (C₁-C₄)alkyl or unsubstituted (C₃-C₇)cycloalkyl; or R⁵ is phenyl(C₁-C₄)alkyl, or a bicyclic ring consisting of two fused five and/or six membered partially saturated, fully saturated or fully unsaturated rings taken independently having one to four heteroatoms selected independently from nitrogen, sulfur and oxygen, said R⁵ substituents optionally substituted on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; or 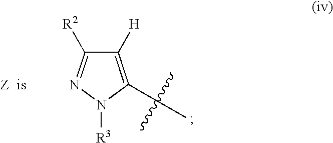
wherein R² is (C₁-C₄)alkyl, (C₃-C₇)cycloalkyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; and
R³ is (C₁-C₄)alkyl, (C₃-C₇)cycloalkyl, phenyl or phenyl(C₁-C₄)alkyl, said (C₁-C₄)alkyl optionally substituted with from one to nine fluorines, said R³ substituent optionally mono- or di-substituted independently with (C₁-C₄)alkoxy, (C₁-C₄)alkylthio, (C₁-C₄)alkylsulfinyl, (C₁-C₄)alkylsulfonyl or (C₁-C₄)alkyl with the proviso that no individual ring of a bicyclic ring can have more than three heteroatoms within the ring.

14. A method of any one of claims 1-12 wherein said compound is:
[5-methyl-1-(quinolin-6-yl)-1H-pyrazole-4-carbonyl]guanidine;
[5-methyl-1-(naphthalen-1-yl)-1H-pyrazole-4-carbonyl]guanidine;
[5-cyclopropyl-1-(quinolin-5-yl)-1H-pyrazole-4-carbonyl]guanidine;
[5-cyclopropyl-1-(quinolin-8-yl)-1H-pyrazole-4-carbonyl]guanidine;
[5-methyl-2-phenyl-2 H-1,2,3-triazole-4-carbonyl]guanidine;
[5-methyl-2-(3-methoxyphenyl)-2H-1 ,2,3-triazole-4-carbonyl]guanidine;
[2-(3-bromophenyl)-5-methyl-2H-1 ,2,3-triazole-4-carbonyl]guanidine;
[5-cyclopropyl-1-(2-trifluoromethylphenyl)-1H-pyrazole-4-carbonyl]guanidine;
[5-cyclopropyl-1-phenyl-1H-pyrazole-4-carbonyl]guanidine;
[5-cyclopropyl-1-(2,6-dichlorophenyl)-1H-pyrazole-4-carbonyl]guanidine;
[1-(2-chloro-4-methylsulfonylphenyl)-5-cyclopropyl-1H-pyrazole-4-carbonyl]guanidine;
[1-(2-chlorophenyl)-5-cyclopropyl-1H-pyrazole-4-carbonyl]guanidine;
[1-(2-trifluoromethyl-4-fluorophenyl)-5-cyclopropyl-1H-pyrazole-4-carbonyl]guanidine;
[1-(2-bromophenyl)-5-cyclopropyl-1H-pyrazole-4-carbonyl]guanidine;
[1-(2-fluorophenyl)-5-cyclopropyl-1H-pyrazole-4-carbonyl]guanidine;
[1-(2-chloro-5-methoxyphenyl)-5-cyclopropyl-1H-pyrazole-4-carbonyl]guanidine;
[1-(2-chloro-4-methylaminosulfonylphenyl)-5-cyclopropyl-1H-pyrazole-4-carbonyl]guanidine;
[1-(2,5-dichlorophenyl)-5-cyclopropyl-1H-pyrazole-4-carbonyl]guanidine;
[1-(2,3-dichlorophenyl)-5-cyclopropyl-1H-pyrazole-4-carbonyl]guanidine;
[1-(2-chloro-5-aminocarbonylphenyl)-5-cyclopropyl-1H-pyrazole-4-carbonyl]guanidine;
[1-(2-chloro-5-aminosulfonylphenyl)-5-cyclopropyl-1H-pyrazole-4-carbonyl]guanidine;
[1-(2-fluoro-6-trifluoromethylphenyl)-5-cyclopropyl-1H-pyrazole-4-carbonyl]guanidine;
[1-(2-chloro-5-methylsulfonylphenyl)-5-cyclopropyl-1H-pyrazole-4-carbonyl]guanidine;
[1-(2-chloro-5-dimethylaminosulfonylphenyl)-5-cyclopropyI-1H-pyrazole-4-carbonyl]guanidine;
[1-(2-trifluoromethyl-4-chlorophenyl)-5-cyclopropyl-1H-pyrazole-4-carbonyl]guanidine;
[1-(8-bromoquinolin-5-yl)-5-cyclopropyl-1H-pyrazole-4-carbonyl]guanidine;
[1-(6-chloroquinolin-5-yl)-5-cyclopropyl-1H-pyrazole-4-carbonyl]guanidine;
[1-(indazol-7-yI)-5-cyclopropyl-1H-pyrazole-4-carbonyl]guanidine;
[1-(benzimidazol-5-yl)-5-cyclopropyl-1H-pyrazole-4-carbonyl]guanidine;
[1-(1-isoquinolyl)-5-cyclopropyl-1H-pyrazole-4-carbonyl]guanidine;
[5-cyclopropyl-1-(4-quinolinyl)-1H-pyrazole-4-carbonyl]guanidine;
[1-(indazol-6-yl)-5-ethyl-1H-pyrazole-4-carbonyl]guanidine;
[1-(indazol-5-yl)-5-ethyl-1H-pyrazole-4-carbonyl]guanidine;
[1-(benzimidazol-5-yI)-5-ethyl-1H-pyrazole-4-carbonyl]guanidine;
[1-(1-methylbenzimidazol-6-yl)-5-ethyl-1H-pyrazole-4-carbonyl]guanidine
[1-(5-quinolinyl)-5-n-propyl-1H-pyrazole-4-carbonyl]guanidine;
[1-(5-quinolinyl)-5-isopropyl-1H-pyrazole-4-carbonyl]guanidine;
[5-ethyl-1-(6-quinolinyl)-1H-pyrazole-4-carbonyl]guanidine;
[1-(2-methylbenzimidazol-5-yl)-5-ethyl-1H-pyrazole-4-carbonyl]guanidine;
[1-(1 ,4-benzodioxan-6-yl)-5-ethyl-1H-pyrazole-4-carbonyl]guanidine;
[1-(benzotriazol-5-yl)-5-ethyl-1H-pyrazole-4-carbonyl]guanidine;
[1-(3-chloroindazol-5-yl)-5-ethyl-1H-pyrazole-4-carbonyl]guanidine;
[1-(5-quinolinyl)-5-butyl-1H-pyrazole-4-carbonyl]guanidine;
[5-propyl-1-(6-quinolinyl)-1H-pyrazole-4-carbonyl]guanidine;
[5-isopropyl-1-(6-quinolinyl)-1H-pyrazole-4-carbonyl]guanidine;
[1-(2-chlorophenyl)-5-methyl-1H-pyrazole-4-carbonyl]guanidine;
[5-methyl-1-(2-trifluoromethylphenyl)-1H-pyrazole-4-carbonyl]guanidine;
[5-ethyl-1-phenyl-1H-pyrazole-4-carbonyl]guanidine;
[5-cyclopropyl-1-(2-trifluoromethylphenyl)-1H-pyrazole-4-carbonyl]guanidine;
[5-cyclopropyl-1-phenyl-1H-pyrazole-4-carbonyl]guanidine; or
[5-cyclopropyl-1-(2,6-dichlorophenyl)-1H-pyrazole-4-carbonyl]guanidine.

15. A method of any one of claims 1-12 wherein said compound is [5-cyclopropyl-1-(quinolin-5-yl)-1H-pyrazole-4-carbonyl]guanidine;
[5-cyclopropyl-1-(2-trifluoromethylphenyl)-1H-pyrazole-4-carbonyl]guanidine;
[1-(2-trifluoromethyl-4-chlorophenyl)-5-cyclopropyl-1H-pyrazole-4-carbonyl]guanidine; or [5-cyclopropyl-1-(2-oxo-1,2-dihydro-quinolin-5-yl)-1H-pyrazole-4-carbonyl]guanidine.

16. A method of treating insulin resistance syndrome (IRS), type 2 diabetes, diabetic neuropathy, diabetic retinopathy, diabetic cardiomyopathy, diabetic microangiopathy, diabetic macroangiopathy, cataracts or foot ulcers in a mammal comprising administering to said mammal a combination of a first pharmaceutical agent, a prodrug or solvate of said first pharmaceutical agent or a pharmaceutically acceptable salt of said first pharmaceutical agent, said prodrug or said solvate of said first pharmaceutical agent and a second pharmaceutical agent, a solvate of said second pharmaceutical agent or a pharmaceutically acceptable salt of said second pharmaceutical agent or said solvate of said second pharmaceutical agent, wherein:
said first pharmaceutical agent is a compound of Formula I: 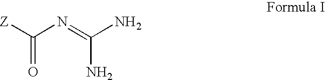
wherein:
Z is carbon connected and is a five-membered, diaza, diunsaturated ring having two contiguous nitrogens, said ring optionally mono-, di-, or tri-substituted with up to three substituents independently selected from R¹, R² and R³; or
Z is carbon connected and is a five-membered, triaza, diunsaturated ring, said ring optionally mono- or di-substituted with up to two substituents independently selected from R⁴ and R⁵;
wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; and
said second pharmaceutical agent is a(n) sulfonyl urea, biguanide, PPAR_{γ} agonist, RXR agonist, α-glucosidase inhibitor, β-agonist, aldose reductase inhibitor, glycogen phosphorylase inhibitor, sorbitol dehydrogenase inhibitor, insulin, insulin analog, insulin secretagogue, vanadate complex, peroxyvanadate, α2-agonist, fatty acid oxidation inhibitor, growth hormone secretagogue, growth hormone mimetic, lipid lowering agent, amylin, amylin antagonist, lipoxygenase inhibitor, antilipolytic agent, somatostatin analog, glucagon antagonist, insulin signaling agonist, insulin mimetic, PTP1 B inhibitor, DPPIV inhibitor, CB-1 receptor antagonist, aP2 inhibitor, SHIP2 inhibitor, gluconeogenesis inhibitor, insulin degrading enzyme inhibitor, cAMP phosphodiesterase inhibitor, cGMP phosphodiesterase inhibitor, glucose transport stimulating agent, glycogen synthase kinase inhibitor, MTP inhibitor, NPY inhibitor, anorectic agent, 5-HT2C receptor agonist, 5-HT2C receptor mimetic, 5HT receptor agonist, 5-HT receptor mimetic, CCKA agonist, serotonin reuptake inhibitor, galanin receptor antagonist, MCR-4 agonist, leptinmimetic, thyromimetic, 11-β-hydroxysteroid dehydrogenase type-1 inhibitor, glucocorticoid receptor antagonist, urocortinmimetic, CRF antagonist or CRF binding protein.

17. A method of treating prophylactically an individual in whom Type 2 diabetes has not yet presented, but in whom there is an increased risk of developing such a condition comprising administering to said mammal a combination of a first pharmaceutical agent, a prodrug or solvate of said first pharmaceutical agent or a pharmaceutically acceptable salt of said first pharmaceutical agent, prodrug or said solvate of said first pharmaceutical agent and a second pharmaceutical agent, a solvate of said second pharmaceutical agent or a pharmaceutically acceptable salt of said second pharmaceutical agent or said solvate of said second pharmaceutical agent, wherein:
said first pharmaceutical agent is a compound of Formula I: 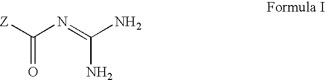
wherein:
Z is carbon connected and is a five-membered, diaza, diunsaturated ring having two contiguous nitrogens, said ring optionally mono-, di-, or tri-substituted with up to three substituents independently selected from R¹, R² and R³; or
Z is carbon connected and is a five-membered, triaza, diunsaturated ring, said ring optionally mono- or di-substituted with up to two substituents independently selected from R⁴ and R⁵;
wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄)cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;
wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇)cycloalkenyl,
wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄)alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines; and
said second pharmaceutical agent is a(n) sulfonyl urea, biguanide, PPAR_{γ} agonist, RXR agonist, α-glucosidase inhibitor, β-agonist, aldose reductase inhibitor, glycogen phosphorylase inhibitor, sorbitol dehydrogenase inhibitor, insulin, insulin analog, insulin secretagogue, vanadate complex, peroxyvanadate, α2-agonist, fatty acid oxidation inhibitor, growth hormone secretagogue, growth hormone mimetic, lipid lowering agent, amylin, amylin antagonist, lipoxygenase inhibitor, antilipolytic agent, somatostatin analog, glucagon antagonist, insulin signaling agonist, insulin mimetic, PTP1 B inhibitor, DPPIV inhibitor, CB-1 receptor antagonist, aP2 inhibitor, SHIP2 inhibitor, gluconeogenesis inhibitor, insulin degrading enzyme inhibitor, cAMP phosphodiesterase inhibitor, cGMP phosphodiesterase inhibitor, glucose transport stimulating agent, glycogen synthase kinase inhibitor, MTP inhibitor, NPY inhibitor, anorectic agent, 5-HT2C receptor agonist, 5-HT2C receptor mimetic, 5HT receptor agonist, 5-HT receptor mimetic, CCKA agonist, serotonin reuptake inhibitor, galanin receptor antagonist, MCR-4 agonist, leptinmimetic, thyromimetic, 11-β-hydroxysteroid dehydrogenase type-1 inhibitor, glucocorticoid receptor antagonist, urocortinmimetic, CRF antagonist or CRF binding protein.

18. A kit comprising:
a) a first unit dosage form comprising a compound of Formula I, a prodrug or solvate thereof or a pharmaceutically acceptable salt of said compound of Formula I, said prodrug or said solvate and a pharmaceutically acceptable carrier, vehicle or diluent;
b) a second unit dosage form comprising:
a(n) sulfonyl urea, biguanide, PPAR_{γ} agonist, RXR agonist, α-glucosidase inhibitor, β-agonist, aldose reductase inhibitor, glycogen phosphorylase inhibitor, sorbitol dehydrogenase inhibitor, insulin, insulin analog, insulin secretagogue, vanadate complex, peroxyvanadate, α2-agonist, fatty acid oxidation inhibitor, growth hormone secretagogue, growth hormone mimetic, lipid lowering agent, amylin, amylin antagonist, lipoxygenase inhibitor, antilipolytic agent, somatostatin analog, glucagon antagonist, insulin signaling agonist, insulin mimetic, PTP1 B inhibitor, DPPIV inhibitor, CB-1 receptor antagonist, aP2 inhibitor, SHIP2 inhibitor, gluconeogenesis inhibitor, insulin degrading enzyme inhibitor, cAMP phosphodiesterase inhibitor, cGMP phosphodiesterase inhibitor, glucose transport stimulating agent, glycogen synthase kinase inhibitor, MTP inhibitor, NPY inhibitor, anorectic agent, 5-HT2C receptor agonist, 5-HT2C receptor mimetic, 5HT receptor agonist, 5-HT receptor mimetic, CCKA agonist, serotonin reuptake inhibitor, galanin receptor antagonist, MCR-4 agonist, leptinmimetic, thyromimetic, 11-β-hydroxysteroid dehydrogenase type-1 inhibitor, glucocorticoid receptor antagonist, urocortinmimetic, CRF antagonist or CRF binding protein; a solvate thereof or a pharmaceutically acceptable salt thereof or of said solvate and a pharmaceutically acceptable carrier, vehicle or diluent; and
c) a container.
